(19) United States
(12) Patent Application Publication
(10) Pub. No.: US 2010/0021869 A1
(43) Pub. Date: Jan. 28, 2010

(54) (METH)ACRYLOYL-CONTAINING MATERIALS, COMPOSITIONS, AND METHODS

(76) Inventors: Ahmed S. Abuelyaman, Woodbury, MN (US); Sumita B. Mitra, West St. Paul, MN (US)

Correspondence Address: 3M INNOVATIVE PROPERTIES COMPANY, PO BOX 33427, ST. PAUL, MN, 55133-3427, US

(21) Appl. No.: 12/520,891

(22) PCT Filed: Dec. 12, 2007
(86) PCT No.: PCT/US07/87202

§ 371 Date: Jun. 23, 2009

Related U.S. Application Data
(60) Provisional application No. 60/877,527, filed on Dec. 28, 2006.

(51) Int. Cl. A61K 6/087; C07C 69/007; A61C 5/00

(52) U.S. Cl. 433/228.1; 523/116; 560/89

(57) ABSTRACT

Di-, tri-, and/or tetra-(meth)acryloyl-containing materials and compositions are provided. Such materials and compositions can preferably be hardened, without undue shrinkage, to provide hardened materials and/or compositions with properties useful, for example, in dental applications. In one aspect, the present invention provides a compound of the formula (Formula I) wherein: each X independently represents an oxygen atom (O) or a nitrogen atom (N); Y and A each independently represent an organic group, with the proviso that Y does not represent —NHCH₂CH₂— if (i) p=O and R¹ represents —C(O)C(CH₃)═CH₂, and/or (ii) q=0 and R² represents —C(O)C(CH₃)═CH₂; m=1 to 5; n=0 to 5; p and q are independently 0 or 1; and R¹ and R² each independently represent H, —C(O)CH═CH₂, or —C(O)C(CH₃)═CH₂.

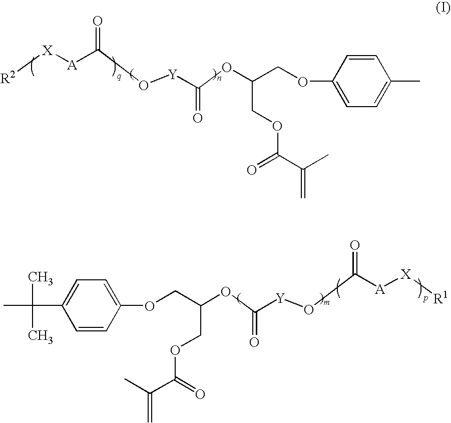

This application claims the benefit of U.S. Provisional Application Ser. No. 60/877,527, filed Dec. 28, 2006, which is incorporated herein by reference in its entirety.

### BACKGROUND

Dental composites, which are made from organic resins and fillers, are finding increasing interest and utility in dental applications due, among other reasons, to their aesthetic properties. Organic resins are known to shrink upon hardening (e.g., crosslinking). As a result, unwanted or excessive shrinkage upon hardening remains as a potential problem that a practitioner may encounter when using such dental composites. For example, excessive shrinkage can cause gaps between the composite and the tooth structure, which can subsequently lead to problems including, for example, post-operative sensitivity, microleakage, and secondary caries.

Significant efforts have been made in attempts to reduce the shrinkage encountered upon hardening dental composites. For example, organic resins used in dental composites typically include di-functional methacrylate resins. Higher functionality (e.g., tri-functional) methacrylate resins have been prepared that show lower shrinkage upon hardening. However, the use of such materials has been hampered, for example, by the difficult and/or expensive methods required to prepare such materials. Further, higher functionality methacrylate resins often have higher viscosities, which can create problems during use, particularly at higher filler loadings.

There remains a need for dental composites that have reduced shrinkage upon hardening.

### SUMMARY

In one aspect, the present invention provides a method of preparing a hardenable material such as a hardenable dental resin. In one embodiment, the method includes: combining components including 2,2-bis[4-(2-hydroxy-3-methacryloxypropoxy)phenyl]propane (BisGMA) and at least one lactone (e.g., ε-caprolactone) under conditions sufficient to chain extend at least one of the two BisGMA hydroxyl groups with a ring-opened lactone. Preferably, the chain-extended BisGMA is non-crystalline. In some embodiments, the method further includes (meth)acryloylating at least one of the BisGMA hydroxyl groups (i.e., chain extended or non-chain extended hydroxyl groups), preferably using at least one (meth)acryloyl-functional acylating agent to provide a tri-(meth)acryloyl-containing material, a tetra-(meth)acryloyl-containing material, or combinations thereof. Preferably, the (meth)acryloylated, chain-extended BisGMA is non-crystalline.

In another aspect, the present invention provides a hardenable composition (e.g., a hardenable dental resin or a hardenable dental composite) including a non-crystalline, chain-extended 2,2-bis[4-(2-hydroxy-3-methacryloxypropoxy)phenyl]propane (BisGMA), wherein the chain extended BisGMA includes at least one of the two BisGMA hydroxyl groups chain-extended with at least one ring-opened lactone (e.g., ring-opened ε-caprolactone). Optionally, hardenable compositions including the hardenable material can further include, for example, an initiator system, an ethylenically unsaturated compound different than the non-crystalline, chain-extended BisGMA, and/or one or more fillers. Also provided are methods of hardening such hardenable compositions, and the resulting hardened compositions.

In another aspect, the present invention provides a method of preparing a hardenable material such as a hardenable dental resin. In one embodiment, the method includes: combining components including 2,2-bis[4-(2-hydroxy-3-methacryloxypropoxy)phenyl]propane (BisGMA) and at least one methacrylate-functional carboxylic acylating agent under conditions sufficient to acylate at least one of the two BisGMA hydroxyl groups. For embodiments in which only one hydroxyl group has been acylated, the remaining unreacted hydroxyl group can optionally be (meth)acryloylated.

In another aspect, the present invention provides a hardenable composition (e.g., a hardenable dental resin or a hardenable dental composite) including an acylated 2,2-bis[4-(2-hydroxy-3-methacryloxypropoxy)phenyl]propane (BisGMA), wherein the acylated BisGMA includes at least one BisGMA hydroxyl group acylated with at least one methacrylate-functional carboxylic acylating agent. Optionally, hardenable compositions including the hardenable material can further include, for example, an initiator system, an ethylenically unsaturated compound different than the acylated BisGMA, and/or one or more fillers. Also provided are methods of hardening such hardenable compositions, and the resulting hardened compositions.

In another aspect, the present invention provides a compound of the formula

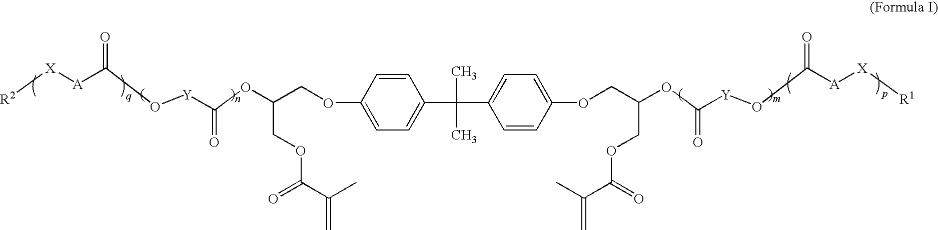

wherein: each X independently represents an oxygen atom (O) or a nitrogen atom (N); Y and A each independently represent an organic group, with the proviso that Y does not represent —NHCH₂CH₂— if (i) p=0 and R¹ represents —C(O)C(CH₃)═CH₂, and/or (ii) q=0 and R² represents —C(O)C(CH₃)═CH₂; m=1 to 5; n=0 to 5; p and q are independently 0 or 1; and R¹ and R² each independently represent H, —C(O)CH═CH₂, or —C(O)C(CH₃)═CH₂. Also provided are hardenable compositions including a compound according to the formula (Formula I). Such hardenable compositions can further include, for example, an initiator system, an ethylenically unsaturated compound different than the compound of the formula (Formula I), and/or one or more fillers. Also provided are methods of hardening such hardenable compositions, and the resulting hardened compositions.

### Definitions

As used herein, “dental composition” refers to an unfilled or filled (e.g. a composite) material (e.g., a dental or orthodontic material) capable of adhering (e.g., bonding) to an oral surface. Dental compositions include, for example, adhesives (e.g., dental and/or orthodontic adhesives), cements (e.g., glass ionomer cements, resin-modified glass ionomer cements, and/or orthodontic cements), primers (e.g., orthodontic primers), restoratives, liners, sealants (e.g., orthodontic sealants), and coatings. Oftentimes a dental composition can be used to bond a dental article to a tooth structure.

As used herein, “dental article” refers to an article that can be adhered (e.g., bonded) to a tooth structure. Dental articles include, for example, crowns, bridges. veneers, inlays, onlays, fillings, orthodontic appliances and devices, and prostheses (e.g., partial or full dentures).

As used herein, “orthodontic appliance” refers to any device intended to be bonded to a tooth structure, including, but not limited to, orthodontic brackets, buccal tubes, lingual retainers, orthodontic bands, bite openers, buttons, and cleats. The appliance has a base for receiving adhesive and it can be a flange made of metal, plastic, ceramic, or combinations thereof. Alternatively, the base can be a custom base formed from cured adhesive layer(s) (i.e., single or multi-layer adhesives).

As used herein, an “oral surface” refers to a soft or hard surface in the oral environment. Hard surfaces typically include tooth structure including, for example, natural and artificial tooth surfaces, bone, tooth models, and the like.

As used herein, “hardenable” is descriptive of a material or composition that can be cured (e.g., polymerized or crosslinked) or solidified, for example, by removing solvent (e.g., by evaporation and/or heating); heating to induce polymerization and/or crosslinking; irradiating to induce polymerization and/or crosslinking; and/or by mixing one or more components to induce polymerization and/or crosslinking. “Mixing” can be performed, for example, by combining two or more parts and mixing to form a homogeneous composition. Alternatively, two or more parts can be provided as separate layers that intermix (e.g., spontaneously or upon application of shear stress) at the interface to initiate polymerization.

As used herein, “hardened” refers to a material or composition that has been cured (e.g., polymerized or crosslinked) or solidified.

As used herein, “hardener” refers to something that initiates hardening of a resin. A hardener may include, for example, a polymerization initiator system, a photoinitiator system, and/or a redox initiator system.

As used herein, “photobleachable” refers to loss of color upon exposure to actinic radiation.

As used herein, the term “(meth)acrylate” is a shorthand reference to acrylate, methacrylate, or combinations thereof; “(meth)acrylic” is a shorthand reference to acrylic, methacrylic, or combinations thereof; and “(meth)acryloyl” is a shorthand reference to acryloyl, methacryloyl, or combinations thereof. As used herein, “(meth)acryloyl-containing compounds” are compounds that include, among other things, a (meth)acrylate moiety, a (meth)acrylamide moiety, or combinations thereof.

The terms “comprises” and variations thereof do not have a limiting meaning where these terms appear in the description and claims.

As used herein, “a,” “an,” “the,” “at least one,” and “one or more” are used interchangeably.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is a graphical representation of measured Watts Shrinkage (%; y-axis) at various time intervals (seconds, x-axis) for the compositions described in Examples 14-16 and Comparative Example 2. The data is also presented in tabular form in Table 5.

FIG. 2 is a graphical representation of measured Watts Shrinkage (%; y-axis) at various time intervals (seconds, x-axis) for the compositions described in Examples 18-21 and Comparative Example 3. The data is also presented in tabular form in Table 8.

### DETAILED DESCRIPTION OF ILLUSTRATIVE EMBODIMENTS

Many dental composites are based on a resin system that includes an aromatic di-methacrylate resin, such as 2,2-bis[4-(2-hydroxy-3-methacryloxypropoxy)phenyl]propane (BisGMA), in combination with a diluent to lower the viscosity of the resin system. Typical diluents, which can facilitate loading of the composite with higher levels of filler, can include low viscosity di-methacrylate monomers, typically a low molecular weight di-methacrylate such as triethyleneglycol dimethacrylate (TEGDMA). However, including low molecular weight methacrylates can often result in excessive shrinkage of the resin system or composite upon hardening.

Some of the materials and compositions disclosed herein can be used in resin systems to provide systems having sufficiently low viscosities to facilitate high filler loading levels in a composite, while exhibiting reduced shrinkage upon hardening. Disclosed herein are di-, tri-, and/or tetra-(meth)acryloyl-containing materials that can be useful, for example, in dental applications. Preferably, at least some of the materials exhibit one or more useful properties including, for example, sufficiently low viscosity to facilitate high filler loading levels in a composite; and reduced shrinkage upon hardening compared, for example, to typically used dental composite resins. In some embodiments, the di-, tri-, and/or tetra-(meth)acryloyl-containing materials disclosed herein can be used as resin systems for dental composites without adding diluents such as low molecular weight di-methacrylates.

The di-, tri-, and/or tetra-(meth)acryloyl-containing materials disclosed herein are preferably reaction products of 2,2-bis[4-(2-hydroxy-3-methacryloxypropoxy)phenyl]propane (BisGMA), in which at least one of the two hydroxyl groups of BisGMA has been reacted with another reactant. Preferably, the di-, tri-, and/or tetra-(meth)acryloyl-containing materials are non-crystalline. A material having a crystalline domain typically exhibits a sharp transition during thermal analysis (e.g., differential thermal analysis, DTA). Thus, “non-crystalline materials,” as used herein, are materials that include no substantial (e.g., detectable) crystalline domain, and thus, no observable sharp transition during thermal analysis (e.g., differential thermal analysis, DTA).

In one embodiment, a reaction product of BisGMA, as disclosed herein, can be formed by chain extending at least one of the two hydroxyl groups of BisGMA with at least one lactone. As used herein, “chain extending” a hydroxyl group with a lactone means that the hydroxyl group has been reacted with at least one lactone molecule, resulting in a chain including the one or more ring-opened lactone molecules being attached to the oxygen atom of the reacted hydroxyl group. As used herein, the “degree” of chain extension refers to the average number of ring-opened lactone molecules in the chains. Such chain extended BisGMA reaction products typically include di-methacrylates having two hydroxyl groups, each of which can be optionally, and independently, further functionalized (e.g., (meth)acryloylated, as further described herein below), to provide, for example, tri- and/or tetra-(meth)acryloyl-containing materials.

In another embodiment, a reaction product of BisGMA, as disclosed herein, can be formed by acylating at least one of the two hydroxyl groups of BisGMA with at least one methacrylate-functional carboxylic acylating agent. As used herein, “acylating” a hydroxyl group means reacting the hydroxyl group with an acylating agent, resulting in a group with a carbonyl being attached to the oxygen atom of the reacted hydroxyl group. As used herein, “carboxylic acylating agents” are acylating agents having a carbonyl group that can react with a hydroxyl group, and include, for example, carboxylic acids, carboxylic acid halides, carboxylic acid anhydrides, activated esters of carboxylic acids (i.e., esters with a good leaving group easily released upon reaction with a hydroxyl group), and combinations thereof. As used herein, “acylating agents” are agents that can react with a hydroxyl group and result in a group having a carbonyl attached to the oxygen atom of the reacted hydroxyl group. Typical acylating agents include, for example, isocyanates, isothiocyanates, carboxylic acids, carboxylic acid halides, carboxylic acid anhydrides, activated esters of carboxylic acids, and combinations thereof. Such acylated BisGMA reaction products typically include tetra-(meth)acryloyl-containing materials having no hydroxyl groups and/or tri-(meth)acryloyl-containing materials having one hydroxyl group, which can optionally be further functionalized (e.g., (meth)acryloylated, as further described herein below) to provide tetra-(meth)acryloyl-containing materials.

In referring to a hydroxyl group, the term “(meth)acryloylating,” as used herein, refers to reacting the hydroxyl group with an agent to provide a (meth)acryloyl-containing group attached to the oxygen atom of the reacted hydroxyl group. Typical (meth)acryloylating agents include, for example, methacrylate-functional acylating agents and (meth)acrylamidating agents.

Useful methacrylate-functional acylating agents include for example, isocyanate-containing materials (e.g., 2-isocyanatoethyl methacrylate) and/or methacrylic acid and derivatives thereof (e.g., a methacryloyl halide, a methacrylic anhydride, an active methacrylic ester such as an ester formed from the reaction of methacrylic acid and N-hydroxy succinimide, and combinations thereof).

Useful (meth)acrylamidating agents includes, for example, 2-alkenylazlactones such as 2-vinyl-4,4-dimethyl-2-oxazolin-5-one (vinyldimethylazlactone, VDMA); 2-isopropenyl-4,4-dimethyl-2-oxazolin-5-one (isopropenyldimethylazlactone, IDMA); and combinations thereof.

In one aspect, disclosed herein are compounds of the formula

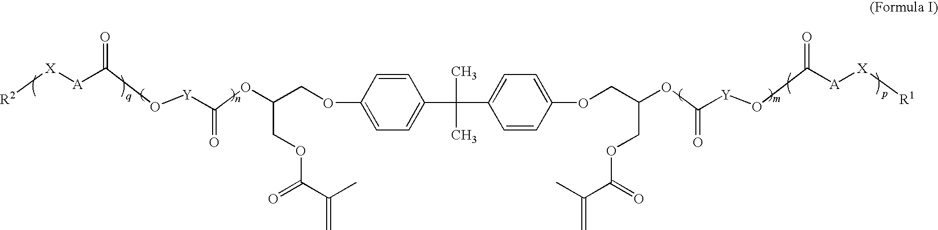

wherein: each X independently represents an oxygen atom (O) or a nitrogen atom (N); Y and A each independently represent an organic group, with the proviso that Y does not represent —NHCH₂CH₂— if (i) p=0 and R¹ represents —C(O)C(CH₃)═CH₂, and/or (ii) q=0 and R² represents —C(O)C(CH₃)═CH₂; m=1 to 5; n=0 to 5; p and q are independently 0 or 1; and R¹ and R² each independently represent H, —C(O)CH═CH₂, or —C(O)C(CH₃)═CH₂. Also provided are hardenable compositions including a compound according to the formula (Formula I). Such hardenable compositions can further include, for example, an initiator system, an ethylenically unsaturated compound different than the compound of the formula (Formula I), and/or one or more fillers. Also provided are methods of hardening such hardenable compositions, and the resulting hardened compositions.

In some preferred embodiments, Y represents —(CH₂)₅—, embodiments which can represent, for example, chain-extension of BisGMA with ε-caprolactone. For some such embodiments, p=q=0, and R¹ and R² can each represent H (e.g., no further functionalization). For some such embodiments, A can represent —NHCH₂CH₂—; preferably p=1, X═O, and R¹ represents —C(O)C(CH₃)═CH₂ (which can represent, for example, functionalization of BisGMA with a methacrylate-functional acylating agent such as 2-isocyantoethyl methacrylate); optionally n=q=0, and R² can represent H (no functionalization of at least one end of BisGMA), or alternatively, when n is not zero, optionally q=1, X═O, and R² can represent —C(O)C(CH₃)═CH₂ (which can represent, for example, functionalization of at least one end of BisGMA with a methacrylate-functional acylating agent such as 2-isocyantoethyl methacrylate).

In some preferred embodiments, Y is selected from the group consisting of —(CH₂)₂C(O)O(CH₂)₂—,

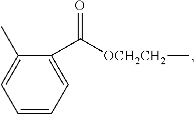

and combinations thereof, embodiments which can represent, for example, acylation of BisGMA with at least one carboxylic acylating agent (e.g., a succinic acid mono-ester, a phthalic acid mono-ester, or combinations thereof). For some such embodiments, preferably m=1, p=0, and R¹ represents —C(O)C(CH₃)═CH₂ (which can represent, for example, acylation of at least one BisGMA hydroxyl group with at least one methacrylate-functional carboxylic acylating agent). For some embodiments, n=q=0, and R² can represent H (no acylation of at least one BisGMA hydroxyl group), or alternatively, n=1, q=0, and R² can represent —C(O)C(CH₃)═CH₂ (which can represent, for example, acylation of at least one BisGMA hydroxyl group with at least one methacrylate-functional carboxylic acylating agent).

As used herein, the term “organic group” is used for the purpose of this invention to mean a hydrocarbon group that is classified as an aliphatic group, cyclic group, or combination of aliphatic and cyclic groups (e.g., alkaryl and aralkyl groups). In the context of the present invention, suitable groups are those, for example, that do not interfere with the hardening of a dental composition or the long-term aging of the composition. In the context of the present invention, the term “aliphatic group” means a saturated or unsaturated linear or branched hydrocarbon group. This term is used to encompass alkyl, alkenyl, and alkynyl groups, for example. The term “alkyl group” means a saturated linear or branched monovalent hydrocarbon group including, for example, methyl, ethyl, n-propyl, isopropyl, tert-butyl, amyl, heptyl, and the like. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.).

As a means of simplifying the discussion and the recitation of certain terminology used throughout this application, the terms “group” and “moiety” (e.g., “organic group” and “organic moiety”) are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, amino, carboxyl, etc. Thus, “alkyl group” includes ether groups, haloalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

### Lactone Chain Extension

In one aspect, compounds of the formula (Formula I) as disclosed herein can provide a hardenable material (e.g., a hardenable dental resin or a hardenable dental composite) including a non-crystalline, chain-extended 2,2-bis[4-(2-hydroxy-3-methacryloxypropoxy)-phenyl]propane (BisGMA), wherein the chain extended BisGMA includes at least one of the two BisGMA hydroxyl groups chain-extended with at least one ring-opened lactone (e.g., ring-opened ε-caprolactone).

Further, methods of preparing such hardenable materials (e.g., a hardenable dental resin) are also provided herein. In one embodiment, the method includes: combining components including 2,2-bis[4-(2-hydroxy-3-methacryloxypropoxy)-phenyl]propane (BisGMA) and at least one lactone under conditions sufficient to chain extend at least one of the two BisGMA hydroxyl groups with a ring-opened lactone. Preferably, the chain-extended BisGMA is non-crystalline. In some embodiments, the method further includes (meth)acryloylating at least one of the chain-extended BisGMA hydroxyl groups, preferably using at least one methacrylate-functional acylating agent and/or at least one (meth)acrylamidating agent to provide a tri-(meth)acryloyl-containing material, a tetra-(meth)acryloyl-containing material, or combinations thereof, as described herein below. Preferably, the (meth)acryloylated, chain-extended BisGMA is non-crystalline.

A wide variety of lactones can be used for chain-extending the hydroxyl group. Such lactones include, for example, ε-caprolactone, γ-butyrolactone, β-propiolactone, and combinations thereof. A particularly preferred lactone includes ε-caprolactone.

Conditions for chain extending a hydroxyl group with one or more ring-opened lactones are well known to one of skill in the art. Typically, the chain extension reaction is run under anhydrous conditions, either neat or including an aprotic solvent, oftentimes under an inert atmosphere (e.g., nitrogen and/or helium). Optionally, a catalyst can be added to facilitate the chain extension reaction. Typical catalysts include, for example, one or more metal-containing catalysts (e.g., tin(II) octanoate) and/or one or more acidic catalysts (e.g., sulfuric acid).

The stoichiometry, mode of addition of reactants (e.g., BisGMA and one or more lactones), and/or reaction conditions can be selected to control the degree of chain extension at each, or both, of the BisGMA hydroxyl groups. For example, equimolar amounts of BisGMA and ε-caprolactone are typically used to substantially chain extend only one of the BisGMA hydroxyl groups. For another example, at least a 2:1 molar ratio of ε-caprolactone:BisGMA is typically used to substantially chain extend both of the BisGMA hydroxyl groups. As used herein, to “substantially chain extend” a BisGMA hydroxyl group means that at least 25%, and in certain embodiments at least 50, 80%, 90%, 95%, 98%, or 99%, and sometimes even 100% of said hydroxyl groups have been chain extended.

For embodiments in which ε-caprolactone is used for chain extending the hydroxyl group, it may be desirable to limit the degree of chain extension to avoid or limit potential crystallinity in the hardenable material. For such embodiments, typically each chain extended hydroxyl group independently includes at most eight ring-opened ε-caprolactone molecules, preferably at most five ring-opened ε-caprolactone molecules, and more preferably at most four ring-opened ε-caprolactone molecules.

Such chain extended BisGMA reaction products typically include di-methacrylates having two hydroxyl groups, each of which can be optionally, and independently, further functionalized (e.g., (meth)acryloylated, as further described herein below), to provide, for example, tri-(meth)acryloyl-containing materials and/or tetra-(meth)acryloyl-containing materials. Thus, in some embodiments, the method further includes (meth)acryloylating at least one of the BisGMA hydroxyl groups (i.e., either chain extended or non-chain extended), preferably using at least one methacrylate-functional acylating agent and/or at least one (meth)acrylamidating agent to provide a tri-(meth)acryloyl-containing material, a tetra-(meth)acryloyl-containing material, or combinations thereof. Preferably, the (meth)acryloylated, chain-extended BisGMA is non-crystalline. (Meth)acryloylating the one or more hydroxyl groups can include, for example, combining components including the chain-extended BisGMA with at least one methacrylate-functional acylating agent and/or at least one (meth)acrylamidating agent under conditions sufficient to form a (meth)acryloylated, chain-extended BisGMA.

A wide variety of methacrylate-functional acylating agents can be used for (meth)acryloylating. Such methacrylate-functional acylating agents include, for example, 2-isocyanatoethyl methacrylate, methacrylic acid, a methacryloyl halide, a methacrylic anhydride, an active methacrylic ester, and combinations thereof.

A wide variety of (meth)acrylamidating agents can also be used for (meth)acryloylating. Such (meth)acrylamidating agents include, for example, 2-alkenylazlactones such as 2-vinyl-4,4-dimethyl-2-oxazolin-5-one (vinyldimethylazlactone, VDMA); 2-isopropenyl-4,4-dimethyl-2-oxazolin-5-one (isopropenyldimethylazlactone, IDMA); and combinations thereof. Conditions for (meth)acryloylating the one or more hydroxyl groups with one or more methacrylate-functional acylating agents and/or (meth)acrylamidating agents are well known to one of skill in the art. Typically, the (meth)acryloylating reaction is run under anhydrous conditions, either neat or including an aprotic solvent, oftentimes under an inert atmosphere (e.g., nitrogen and/or helium).

Optionally, a catalyst can be added to facilitate the (meth)acryloylating reaction. Typical catalysts for (meth)acryloylating using an isocyanate-containing material (e.g., 2-isocyanatoethyl methacrylate) include, for example, organometallic compounds (e.g., dibutyltin dilaurate), Lewis acids (e.g., copper(I) chloride), Bronsted acids (e.g., HCl), and combinations thereof. Typical catalysts for (meth)acryloylating using carboxylic acids include, for example, coupling agents (e.g., dicyclohexylcarbodiimide (DCC), carbonyl diimidazole (CDI), or combinations thereof) in the presence of an amine catalyst (e.g., dimethyl aminopyridine (DMAP)). Typical catalysts for (meth)acryloylating using, for example, carboxylic acid halides (e.g., methacryloyl chloride), carboxylic acid anhydrides (e.g., methacrylic anhydride), and/or activated esters of carboxylic acids (e.g., an ester formed from the reaction of methacrylic acid and N-hydroxy succinimide) include, for example, bases (e.g., triethylamine, pyridine, and combinations thereof.

The stoichiometry, mode of addition of reactants, and/or reaction conditions can be selected to control the degree of (meth)acryloylation at each, or both, of the chain-extended BisGMA hydroxyl groups. For example, equimolar amounts of chain-extended BisGMA and a methacrylate-functional carboxylic acid (e.g., methacrylic acid) in the presence of an equimolar amount of dicyclohexylcarbodiimide (DCC) and a 0.1 molar amount of dimethyl aminopyridine (DMAP) are typically used to substantially (meth)acryloylate only one of the hydroxyl groups. For another example, a 2:1:2 molar ratio of methacrylate-functional carboxylic acid (e.g., methacrylic acid):BisGMA:DCC in the presence of a 0.2 molar amount of DMAP is typically used to substantially (meth)acryloylate both of the hydroxyl groups. As used herein, to “substantially (meth)acryloylate” a hydroxyl group means that at least 25%, and in certain embodiments at least 50, 80%, 90%, 95%, 98%, or 99%, and sometimes even 100% of said hydroxyl groups have been (meth)acryloylated.

### Acylated Materials

In another aspect, compounds of the formula (Formula I) as disclosed herein can provide a hardenable material (e.g., a hardenable dental resin or a hardenable dental composite) including an acylated 2,2-bis[4-(2-hydroxy-3-methacryloxypropoxy)phenyl]propane (BisGMA), wherein the acylated BisGMA includes at least one BisGMA hydroxyl group acylated with at least one methacrylate-functional carboxylic acylating agent. In some embodiments, the carboxylic acylating agent includes a methacrylate-functional carboxylic acid (e.g., mono-2-(methacryloyloxyethyl)-succinate (e.g., to prepare TrisMAS), mono-2-(methacryloyloxyethyl)phthalate (e.g., to prepare TrisMAP), mono-3-(methacryloylxyethyl)glutarate, mono-4-(methacryloyloxyethyl)adipate, and combinations thereof); a methacrylate-functional carboxylic acid halide; a methacrylate-functional carboxylic acid anhydride; an active ester of a methacrylate-functional carboxylic acid; or combinations thereof.

Further, methods of preparing such hardenable materials (e.g., a hardenable dental resin) are also provided herein. In one embodiment, the method includes: combining components including 2,2-bis[4-(2-hydroxy-3-methacryloxypropoxy)-phenyl]propane (BisGMA) and at least one methacrylate-functional carboxylic acylating agent under conditions sufficient to acylate at least one of the two BisGMA hydroxyl groups. For embodiments in which only one hydroxyl group has been acylated, the remaining unreacted hydroxyl group can optionally be (meth)acryloylated preferably using at least one methacrylate-functional acylating agent, to provide a tetra-(meth)acryloyl-containing material, as described herein below.

A wide variety of methacrylate-functional carboxylic acylating agents can be used for acylating the hydroxyl group. Such methacrylate-functional carboxylic acylating agents include, for example, methacrylate-functional carboxylic acids (e.g., a mono-ester of a dicarboxylic acid), methacrylate-functional carboxylic acid halides, methacrylate-functional carboxylic acid anhydrides, methacrylate-functional active esters of carboxylic acids, and combinations thereof. Methacrylate-functional carboxylic acids include, for example, mono-2-(methacryloyloxyethyl)-succinate, mono-2-(methacryloyloxyethyl)phthalate, mono-3-(methacryloylxyethyl)glutarate, mono-4-(methacryloyloxyethyl)adipate, and combinations thereof.

Conditions for acylating a hydroxyl group with one or more methacrylate-functional carboxylic acylating agents are well known to one of skill in the art. Typically, the acylating reaction is run under anhydrous conditions, either neat or including an aprotic solvent, oftentimes under an inert atmosphere (e.g., nitrogen and/or helium).

Optionally, a catalyst can be added to facilitate the acylation reaction. Typical catalysts for acylating using carboxylic acids include, for example, coupling agents (e.g., dicyclohexylcarbodiimide (DCC), carbonyl diimidazole (CDI), or combinations thereof) in the presence of an amine catalyst (e.g., dimethyl aminopyridine (DMAP)). Typical catalysts for acylating using, for example, carboxylic acid halides (e.g., methacryloyl chloride), carboxylic acid anhydrides (e.g., methacrylic anhydride), and/or activated esters of carboxylic acids (e.g., an ester formed from the reaction of methacrylic acid and N-hydroxy succinimide) include, for example, bases (e.g., triethylamine, pyridine, and combinations thereof.

The stoichiometry, mode of addition of reactants (e.g., BisGMA and one or more acylating agents), and/or reaction conditions can be selected to control the degree of acylation at each, or both, of the BisGMA hydroxyl groups. For example, equimolar amounts of BisGMA and a methacrylate-functional carboxylic acid (e.g., mono-2-(methacryloyloxyethyl)-succinate) in the presence of an equimolar amount of dicyclohexylcarbodiimide (DCC) and a 0.1 molar amount of dimethyl aminopyridine (DMAP) are typically used to substantially acylate only one of the BisGMA hydroxyl groups. For another example, a 2:1:2 molar ratio of methacrylate-functional carboxylic acid (e.g., mono-2-(methacryloyloxyethyl)-succinate):BisGMA:DCC in the presence of a 0.2 molar amount of DMAP is typically used to substantially acylate both of the BisGMA hydroxyl groups. As used herein, to “substantially acylate” a BisGMA hydroxyl group means that at least 25%, and in certain embodiments at least 50, 80%, 90%, 95%, 98%, or 99%, and sometimes even 100% of said BisGMA hydroxyl groups have been acylated.

Such acylated BisGMA reaction products typically include tetra-methacrylates having no hydroxyl groups and/or tri-methacrylates having one hydroxyl group, which can optionally be further functionalized (e.g., (meth)acryloylated, as further described herein), to provide, for example, tetra-(meth)acryloyl-containing materials. Thus, in some embodiments, the method further includes (meth)acryloylating a BisGMA hydroxyl group, preferably using at least one methacrylate-functional acylating agent and/or at least one (meth)acrylamidating agent to provide a tetra-(meth)acryloyl-containing material. Methods and conditions for (meth)acryloylating a hydroxyl group using one or more methacrylate functional acylating agents and/or one or more (meth)acrylamidating agents are described herein.

### Compositions

Optionally, hardenable compositions including one or more hardenable materials as described herein can further include, for example, an initiator system, an ethylenically unsaturated compound different than the non-crystalline, chain-extended BisGMA, and/or one or more fillers. Hardenable and hardened dental compositions of the present invention can be used for a variety of dental and orthodontic applications that utilize a material capable of adhering (e.g., bonding) to a tooth structure. Uses for such hardenable and hardened dental compositions include, for example, uses as adhesives (e.g., dental and/or orthodontic adhesives), cements (e.g., glass ionomer cements, resin-modified glass ionomer cements, and orthodontic cements), primers (e.g., orthodontic primers), restoratives, liners, sealants (e.g., orthodontic sealants), coatings, and combinations thereof.

Hardenable compositions (e.g., hardenable dental compositions) as described herein typically include a hardenable (e.g., polymerizable) component, thereby forming hardenable (e.g., polymerizable) compositions. In addition to the hardenable materials described herein, the hardenable component can include a wide variety of chemistries, such as ethylenically unsaturated compounds (with or without acid functionality), epoxy (oxirane) resins, vinyl ethers, photopolymerization systems, redox cure systems, glass ionomer cements, polyethers, polysiloxanes, and the like. In some embodiments, the compositions can be hardened (e.g., polymerized by conventional photopolymerization and/or chemical polymerization techniques) prior to applying the hardened dental composition. In other embodiments, a dental composition can be hardened (e.g., polymerized by conventional photopolymerization and/or chemical polymerization techniques) after applying the dental composition.

In certain embodiments, the compositions are photopolymerizable, i.e., the compositions contain a photoinitiator (i.e., a photoinitiator system) that upon irradiation with actinic radiation initiates the polymerization (or hardening) of the composition. Such photopolymerizable compositions can be free radically polymerizable or cationically polymerizable. In other embodiments, the compositions are chemically hardenable, i.e., the compositions contain a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements (e.g., conventional and resin-modified glass ionomer cements), redox cure systems, and combinations thereof.

Suitable photopolymerizable components that can be used in the dental compositions of the present invention include, for example, epoxy resins (which contain cationically active epoxy groups), vinyl ether resins (which contain cationically active vinyl ether groups), ethylenically unsaturated compounds (which contain free radically active unsaturated groups, e.g., acrylates and methacrylates), and combinations thereof. Also suitable are polymerizable materials that contain both a cationically active functional group and a free radically active functional group in a single compound. Examples include epoxy-functional acrylates, epoxy-functional methacrylates, and combinations thereof.

### Ethylenically Unsaturated Compounds

The compositions of the present invention may include one or more hardenable components in the form of ethylenically unsaturated compounds with or without acid functionality, thereby forming hardenable compositions.

Suitable hardenable compositions may include hardenable components (e.g., photopolymerizable compounds) that include ethylenically unsaturated compounds (which contain free radically active unsaturated groups). Examples of useful ethylenically unsaturated compounds include acrylic acid esters, methacrylic acid esters, hydroxy-functional acrylic acid esters, hydroxy-functional methacrylic acid esters, and combinations thereof.

The compositions (e.g., photopolymerizable compositions) may include compounds having free radically active functional groups that may include monomers, oligomers, and polymers having one or more ethylenically unsaturated group. Suitable compounds contain at least one ethylenically unsaturated bond and are capable of undergoing addition polymerization. Such free radically polymerizable compounds include mono-, di- or poly-(meth)acrylates (i.e., acrylates and methacrylates) such as, methyl(meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenol A di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in PCT International Publication Nos. WO 00/38619 (Guggenberger et al.), WO 01/92271 (Weinmann et al.), WO 01/07444 (Guggenberger et al.), and WO 00/42092 (Guggenberger et al.); and fluoropolymer-functional (meth)acrylates as disclosed, for example, in U.S. Pat. No. 5,076,844 (Fock et al.) and U.S. Pat. No. 4,356,296 (Griffith et al.) and European Pat. Application Publication Nos. EP 0373 384 (Wagenknecht et al.), EP 0201 031 (Reiners et al.), and EP 0201 778 (Reiners et al.). Mixtures of two or more free radically polymerizable compounds can be used if desired.

The hardenable component may also contain hydroxyl groups and ethylenically unsaturated groups in a single molecule. Examples of such materials include hydroxyalkyl(meth)acrylates, such as 2-hydroxyethyl(meth)acrylate and 2-hydroxypropyl(meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-ethacryloxypropoxy)phenyl]propane (bisGMA). Suitable ethylenically unsaturated compounds are also available from a wide variety of commercial sources, such as Sigma-Aldrich, St. Louis. Mixtures of ethylenically unsaturated compounds can be used if desired.

In certain embodiments hardenable components can include PEGDMA (polyethyleneglycol dimethacrylate having a molecular weight of approximately 400), bisGMA, UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). Various combinations of the hardenable components can be used if desired.

In certain embodiments hardenable components can include one or more polymerizable hybrid compounds. Exemplary polymerizable hybrid compounds include, for example, compounds having at least one cyclic allylic sulfide group and at least one (meth)acryloyl group as described, for example, in PCT International Publication No. WO 2006/122081 A1 (Abuelyaman et al.). In some embodiments, polymerizable hybrid compounds can be included in dental compositions that, upon hardening, exhibit low shrinkage along with good mechanical properties.

Preferably, compositions of the present invention include at least 5% by weight, more preferably at least 10% by weight, and most preferably at least 15% by weight ethylenically unsaturated compounds, based on the total weight of the unfilled composition. Preferably, compositions of the present invention include at most 95% by weight, more preferably at most 90% by weight, and most preferably at most 80% by weight ethylenically unsaturated compounds, based on the total weight of the unfilled composition.

Preferably, compositions of the present invention include ethylenically unsaturated compounds without acid functionality. Preferably, compositions of the present invention include at least 5% by weight (wt-%), more preferably at least 10% by weight, and most preferably at least 15% by weight ethylenically unsaturated compounds without acid functionality, based on the total weight of the unfilled composition. Preferably, compositions of the present invention include at most 95% by weight, more preferably at most 90% by weight, and most preferably at most 80% by weight ethylenically unsaturated compounds without acid functionality, based on the total weight of the unfilled composition.

Ethylenically Unsaturated Compounds with Acid Functionality

The compositions of the present invention may include one or more hardenable components in the form of ethylenically unsaturated compounds with acid functionality, thereby forming hardenable compositions.

As used herein, ethylenically unsaturated compounds with acid functionality is meant to include monomers, oligomers, and polymers having ethylenic unsaturation and acid and/or acid-precursor functionality. Acid-precursor functionalities include, for example, anhydrides, acid halides, and pyrophosphates. The acid functionality can include carboxylic acid functionality, phosphoric acid functionality, phosphonic acid functionality, sulfonic acid functionality, or combinations thereof.

Ethylenically unsaturated compounds with acid functionality include, for example, α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl)phosphate, ((meth)acryloxypropyl)phosphate, bis((meth)acryloxypropyl)phosphate, bis((meth)acryloxy)propyloxy phosphate, (meth)acryloxyhexyl phosphate, bis((meth)acryloxyhexyl)phosphate, (meth)acryloxyoctyl phosphate, bis((meth)acryloxyoctyl)phosphate, (meth)acryloxydecyl phosphate, bis((meth)acryloxydecyl)phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components in the hardenable component system. Also monomers, oligomers, and polymers of unsaturated carbonic acids such as (meth)acrylic acids, aromatic (meth)acrylated acids (e.g., methacrylated trimellitic acids), and anhydrides thereof can be used. Certain preferred compositions of the present invention include an ethylenically unsaturated compound with acid functionality having at least one P—OH moiety.

Certain of these compounds are obtained, for example, as reaction products between isocyanatoalkyl(meth)acrylates and carboxylic acids. Additional compounds of this type having both acid-functional and ethylenically unsaturated components are described in U.S. Pat. No. 4,872,936 (Engelbrecht) and U.S. Pat. No. 5,130,347 (Mitra). A wide variety of such compounds containing both the ethylenically unsaturated and acid moieties can be used. Mixtures of such compounds can be used if desired.

Additional ethylenically unsaturated compounds with acid functionality include, for example, polymerizable bisphosphonic acids as disclosed for example, in U.S. Pat. Application Publication No. 2004/0206932 (Abuelyaman et al.); AA:ITA:IEM (copolymer of acrylic acid:itaconic acid with pendent methacrylate made by reacting AA:ITA copolymer with sufficient 2-isocyanatoethyl methacrylate to convert a portion of the acid groups of the copolymer to pendent methacrylate groups as described, for example, in Example 11 of U.S. Pat. No. 5,130,347 (Mitra)); and those recited in U.S. Pat. No. 4,259,075 (Yamauchi et al.), U.S. Pat. No. 4,499,251 (Omura et al.), U.S. Pat. No. 4,537,940 (Omura et al.), U.S. Pat. No. 4,539,382 (Omura et al.), U.S. Pat. No. 5,530,038 (Yamamoto et al.), U.S. Pat. No. 6,458,868 (Okada et al.), and European Pat. Application Publication Nos. EP 712,622 (Tokuyama Corp.) and EP 1,051,961 (Kuraray Co., Ltd.).

Compositions of the present invention can also include compositions that include combinations of ethylenically unsaturated compounds with acid functionality. Preferably the compositions are self-adhesive and are non-aqueous. For example, such compositions can include: a first compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C1-C4 hydrocarbon group; a second compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C5-C12 hydrocarbon group; an ethylenically unsaturated compound without acid functionality; an initiator system; and a filler. Such compositions are described, for example, in PCT International Publication No. WO 2006/020760 A1 (Luchterhandt et al.).

Preferably, the compositions of the present invention include at least 1% by weight, more preferably at least 3% by weight, and most preferably at least 5% by weight ethylenically unsaturated compounds with acid functionality, based on the total weight of the unfilled composition. Preferably, compositions of the present invention include at most 80% by weight, more preferably at most 70% by weight, and most preferably at most 60% by weight ethylenically unsaturated compounds with acid functionality, based on the total weight of the unfilled composition.

### Epoxy (Oxirane) or Vinyl Ether Compounds

The hardenable compositions of the present invention may include one or more hardenable components in the form of epoxy (oxirane) compounds (which contain cationically active epoxy groups) or vinyl ether compounds (which contain cationically active vinyl ether groups), thereby forming hardenable compositions.

The epoxy or vinyl ether monomers can be used alone as the hardenable component in a dental composition or in combination with other monomer classes, e.g., ethylenically unsaturated compounds as described herein, and can include as part of their chemical structures aromatic groups, aliphatic groups, cycloaliphatic groups, and combinations thereof.

Examples of epoxy (oxirane) compounds include organic compounds having an oxirane ring that is polymerizable by ring opening. These materials include monomeric epoxy compounds and epoxides of the polymeric type and can be aliphatic, cycloaliphatic, aromatic or heterocyclic. These compounds generally have, on the average, at least 1 polymerizable epoxy group per molecule, in some embodiments at least 1.5, and in other embodiments at least 2 polymerizable epoxy groups per molecule. The polymeric epoxides include linear polymers having terminal epoxy groups (e.g., a diglycidyl ether of a polyoxyalkylene glycol), polymers having skeletal oxirane units (e.g., polybutadiene polyepoxide), and polymers having pendent epoxy groups (e.g., a glycidyl methacrylate polymer or copolymer). The epoxides may be pure compounds or may be mixtures of compounds containing one, two, or more epoxy groups per molecule. The “average” number of epoxy groups per molecule is determined by dividing the total number of epoxy groups in the epoxy-containing material by the total number of epoxy-containing molecules present.

These epoxy-containing materials may vary from low molecular weight monomeric materials to high molecular weight polymers and may vary greatly in the nature of their backbone and substituent groups. Illustrative of permissible substituent groups include halogens, ester groups, ethers, sulfonate groups, siloxane groups, carbosilane groups, nitro groups, phosphate groups, and the like. The molecular weight of the epoxy-containing materials may vary from 58 to 100,000 or more.

Suitable epoxy-containing materials useful as the resin system reactive components in the present invention are listed in U.S. Pat. No. 6,187,836 (Oxman et al.) and U.S. Pat. No. 6,084,004 (Weinmann et al.).

Other suitable epoxy resins useful as the resin system reactive components include those which contain cyclohexene oxide groups such as epoxycyclohexanecarboxylates, typified by 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexyl-methyl) adipate. For a more detailed list of useful epoxides of this nature, reference is made to U.S. Pat. No. 5,037,861 (Crivello et al.), U.S. Pat. No. 6,245,828 (Weinmann et al.), and U.S. Pat. No. 6,779,656 (Klettke et al.).

Other epoxy resins that may be useful in the compositions of this invention include glycidyl ether monomers. Examples are glycidyl ethers of polyhydric phenols obtained by reacting a polyhydric phenol with an excess of chlorohydrin such as epichlorohydrin (e.g., the diglycidyl ether of 2,2-bis-(2,3-epoxypropoxyphenol)propane). Further examples of epoxides of this type are described in U.S. Pat. No. 3,018,262 (Schroeder), and in “Handbook of Epoxy Resins” by Lee and Neville, McGraw-Hill Book Co., New York (1967).

Other suitable epoxides useful as the resin system reactive components are those that contain silicon, useful examples of which are described in PCT International Publication No. WO 01/51540 (Klettke et al.).

Additional suitable epoxides useful as the resin system reactive components include octadecylene oxide, epichlorohydrin, styrene oxide, vinyl cyclohexene oxide, glycidol, glycidylmethacrylate, diglycidyl ether of Bisphenol A and other commercially available epoxides, as provided in U.S. Pat. Application Publication No. 2005/0113477 A1 (Oxman et al.).

Blends of various epoxy-containing materials are also contemplated. Examples of such blends include two or more weight average molecular weight distributions of epoxy-containing compounds, such as low molecular weight (below 200), intermediate molecular weight (200 to 10,000) and higher molecular weight (above 10,000). Alternatively or additionally, the epoxy resin may contain a blend of epoxy-containing materials having different chemical natures, such as aliphatic and aromatic, or functionalities, such as polar and non-polar.

Other types of useful hardenable components having cationically active functional groups include vinyl ethers, oxetanes, spiro-orthocarbonates, spiro-orthoesters, and the like.

If desired, both cationically active and free radically active functional groups may be contained in a single molecule. Such molecules may be obtained, for example, by reacting a di- or poly-epoxide with one or more equivalents of an ethylenically unsaturated carboxylic acid. An example of such a material is the reaction product of UVR-6105 (available from Union Carbide) with one equivalent of methacrylic acid. Commercially available materials having epoxy and free-radically active functionalities include the CYCLOMER series, such as CYCLOMER M-100, M-101, or A-200 available from Daicel Chemical, Japan, and EBECRYL-3605 available from Radcure Specialties, UCB Chemicals, Atlanta, Ga.

The cationically curable components may further include a hydroxyl-containing organic material. Suitable hydroxyl-containing materials may be any organic material having hydroxyl functionality of at least 1, and preferably at least 2. Preferably, the hydroxyl-containing material contains two or more primary or secondary aliphatic hydroxyl groups (i.e., the hydroxyl group is bonded directly to a non-aromatic carbon atom). The hydroxyl groups can be terminally situated, or they can be pendent from a polymer or copolymer. The molecular weight of the hydroxyl-containing organic material can vary from very low (e.g., 32) to very high (e.g., one million or more). Suitable hydroxyl-containing materials can have low molecular weights (i.e., from 32 to 200), intermediate molecular weights (i.e., from 200 to 10,000, or high molecular weights (i.e., above 10,000). As used herein, all molecular weights are weight average molecular weights.

The hydroxyl-containing materials may be non-aromatic in nature or may contain aromatic functionality. The hydroxyl-containing material may optionally contain heteroatoms in the backbone of the molecule, such as nitrogen, oxygen, sulfur, and the like. The hydroxyl-containing material may, for example, be selected from naturally occurring or synthetically prepared cellulosic materials. The hydroxyl-containing material should be substantially free of groups which may be thermally or photolytically unstable; that is, the material should not decompose or liberate volatile components at temperatures below 100° C. or in the presence of actinic light which may be encountered during the desired photopolymerization conditions for the polymerizable compositions.

Suitable hydroxyl-containing materials useful in the present invention are listed in U.S. Pat. No. 6,187,836 (Oxman et al.).

The hardenable component(s) may also contain hydroxyl groups and cationically active functional groups in a single molecule. An example is a single molecule that includes both hydroxyl groups and epoxy groups.

### Glass Ionomers

The hardenable compositions of the present invention may include glass ionomer cements such as conventional glass ionomer cements that typically employ as their main ingredients a homopolymer or copolymer of an ethylenically unsaturated carboxylic acid (e.g., poly acrylic acid, copoly (acrylic, itaconic acid), and the like), a fluoroaluminosilicate (“FAS”) glass, water, and a chelating agent such as tartaric acid. Conventional glass ionomers (i.e., glass ionomer cements) typically are supplied in powder/liquid formulations that are mixed just before use. The mixture will undergo self-hardening in the dark due to an ionic reaction between the acidic repeating units of the polycarboxylic acid and cations leached from the glass.

The glass ionomer cements may also include resin-modified glass ionomer (“RMGI”) cements. Like a conventional glass ionomer, an RMGI cement employs an FAS glass. However, the organic portion of an RMGI is different. In one type of RMGI, the polycarboxylic acid is modified to replace or end-cap some of the acidic repeating units with pendent curable groups and a photoinitiator is added to provide a second cure mechanism, e.g., as described in U.S. Pat. No. 5,130,347 (Mitra). Acrylate or methacrylate groups are usually employed as the pendant curable group. In another type of RMGI, the cement includes a polycarboxylic acid, an acrylate or methacrylate-functional monomer and a photoinitiator, e.g., as in Mathis et al., “Properties of a New Glass Ionomer/Composite Resin Hybrid Restorative”, Abstract No. 51, J. Dent Res., 66:113 (1987) and as in U.S. Pat. No. 5,063,257 (Akahane et al.), U.S. Pat. No. 5,520,725 (Kato et al.), U.S. Pat. No. 5,859,089 (Qian), U.S. Pat. No. 5,925,715 (Mitra) and U.S. Pat. No. 5,962,550 (Akahane et al.). In another type of RMGI, the cement may include a polycarboxylic acid, an acrylate or methacrylate-functional monomer, and a redox or other chemical cure system, e.g., as described in U.S. Pat. No. 5,154,762 (Mitra et al.), U.S. Pat. No. 5,520,725 (Kato et al.), and U.S. Pat. No. 5,871,360 (Kato). In another type of RMGI, the cement may include various monomer-containing or resin-containing components as described in U.S. Pat. No. 4,872,936 (Engelbrecht), U.S. Pat. No. 5,227,413 (Mitra), U.S. Pat. No. 5,367,002 (Huang et al.), and U.S. Pat. No. 5,965,632 (Orlowski). RMGI cements are preferably formulated as powder/liquid or paste/paste systems, and contain water as mixed and applied. The compositions are able to harden in the dark due to the ionic reaction between the acidic repeating units of the polycarboxylic acid and cations leached from the glass, and commercial RMGI products typically also cure on exposure of the cement to light from a dental curing lamp. RMGI cements that contain a redox cure system and that can be cured in the dark without the use of actinic radiation are described in U.S. Pat. No. 6,765,038 (Mitra).

### Photoinitiator Systems

In certain embodiments, the compositions of the present invention are photopolymerizable, i.e., the compositions contain a photopolymerizable component and a photoinitiator (i.e., a photoinitiator system) that upon irradiation with actinic radiation initiates the polymerization (or hardening) of the composition. Such photopolymerizable compositions can be free radically polymerizable or cationically polymerizable.

Suitable photoinitiators (i.e., photoinitiator systems that include one or more compounds) for polymerizing free radically photopolymerizable compositions include binary and tertiary systems. Typical tertiary photoinitiators include an iodonium salt, a photosensitizer, and an electron donor compound as described in U.S. Pat. No. 5,545,676 (Palazzotto et al.). Preferred iodonium salts are the diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Preferred photosensitizers are monoketones and diketones that absorb some light within a range of 400 nm to 520 nm (preferably, 450 nm to 500 nm). More preferred compounds are alpha diketones that have some light absorption within a range of 400 nm to 520 nm (even more preferably, 450 to 500 nm). Preferred compounds are camphorquinone, benzil, furil, 3,3,6,6-tetramethylcyclohexanedione, phenanthraquinone, 1-phenyl-1,2-propanedione and other 1-aryl-2-alkyl-1,2-ethanediones, and cyclic alpha diketones. Most preferred is camphorquinone. Preferred electron donor compounds include substituted amines, e.g., ethyl dimethylaminobenzoate. Other suitable tertiary photoinitiator systems useful for photopolymerizing cationically polymerizable resins are described, for example, in U.S. Pat. No. 6,765,036 (Dede et al.).

Other suitable photoinitiators for polymerizing free radically photopolymerizable compositions include the class of phosphine oxides that typically have a functional wavelength range of 380 nm to 1200 nm. Preferred phosphine oxide free radical initiators with a functional wavelength range of 380 nm to 450 nm are acyl and bisacyl phosphine oxides such as those described in U.S. Pat. No. 4,298,738 (Lechtken et al.), U.S. Pat. No. 4,324,744 (Lechtken et al.), U.S. Pat. No. 4,385,109 (Lechtken et al.), U.S. Pat. No. 4,710,523 (Lechtken et al.), and U.S. Pat. No. 4,737,593 (Ellrich et al.), U.S. Pat. No. 6,251,963 (Kohler et al.); and European Pat. Application Publication No. 0 173 567 A2 (Ying).

Commercially available phosphine oxide photoinitiators capable of free-radical initiation when irradiated at wavelength ranges of greater than 380 nm to 450 nm include bis(2,4,6-trimethylbenzoyl)phenyl phosphine oxide (IRGACURE 819, Ciba Specialty Chemicals, Tarrytown, N.Y.), bis(2,6-dimethoxybenzoyl)-(2,4,4-trimethylpentyl)phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1-phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF Corp., Charlotte, N.C.).

Typically, the phosphine oxide initiator is present in the photopolymerizable composition in catalytically effective amounts, such as from 0.1 weight percent to 5.0 weight percent, based on the total weight of the composition.

Tertiary amine reducing agents may be used in combination with an acylphosphine oxide. Illustrative tertiary amines useful in the invention include ethyl 4-(N,N-dimethylamino)benzoate and N,N-dimethylaminoethyl methacrylate. When present, the amine reducing agent is present in the photopolymerizable composition in an amount from 0.1 weight percent to 5.0 weight percent, based on the total weight of the composition. Useful amounts of other initiators are well known to those of skill in the art.

Suitable photoinitiators for polymerizing cationically photopolymerizable compositions include binary and tertiary systems. Typical tertiary photoinitiators include an iodonium salt, a photosensitizer, and an electron donor compound as described in EP 0 897 710 (Weinmann et al.); in U.S. Pat. No. 5,856,373 (Kaisaki et al.), U.S. Pat. No. 6,084,004 (Weinmann et al.), U.S. Pat. No. 6,187,833 (Oxman et al.), and U.S. Pat. No. 6,187,836 (Oxman et al.); and in U.S. Pat. No. 6,765,036 (Dede et al.). The compositions of the invention can include one or more anthracene-based compounds as electron donors. In some embodiments, the compositions comprise multiple substituted anthracene compounds or a combination of a substituted anthracene compound with unsubstituted anthracene. The combination of these mixed-anthracene electron donors as part of a photoinitiator system provides significantly enhanced cure depth and cure speed and temperature insensitivity when compared to comparable single-donor photoinitiator systems in the same matrix. Such compositions with anthracene-based electron donors are described in U.S. Pat. Application Publication No. 2005/0113477 A1 (Oxman et al.).

Suitable iodonium salts include tolylcumyliodonium tetrakis(pentafluorophenyl)borate, tolylcumyliodonium tetrakis(3,5-bis(trifluoromethyl)-phenyl)borate, and the diaryl iodonium salts, e.g., diphenyliodonium chloride, diphenyliodonium hexafluorophosphate, diphenyliodonium hexafluoroantimonate, and diphenyliodonium tetrafluoroborate. Suitable photosensitizers include monoketones and diketones that absorb some light within a range of 450 nm to 520 nm (preferably, 450 nm to 500 nm). More suitable compounds include alpha diketones that have some light absorption within a range of 450 nm to 520 nm (even more preferably, 450 nm to 500 nm). Preferred compounds include camphorquinone, benzil, furil, 3,3,6,6-tetramethylcyclohexanedione, phenanthraquinone and other cyclic alpha diketones. Most preferred is camphorquinone. Suitable electron donor compounds include substituted amines, e.g., ethyl 4-(dimethylamino)benzoate and 2-butoxyethyl 4-(dimethylamino)benzoate; and polycondensed aromatic compounds (e.g. anthracene).

The initiator system is present in an amount sufficient to provide the desired rate of hardening (e.g., polymerizing and/or crosslinking). For a photoinitiator, this amount will be dependent in part on the light source, the thickness of the layer to be exposed to radiant energy, and the extinction coefficient of the photoinitiator. Preferably, the initiator system is present in a total amount of at least 0.01 wt-%, more preferably, at least 0.03 wt-%, and most preferably, at least 0.05 wt-%, based on the weight of the composition. Preferably, the initiator system is present in a total amount of no more than 10 wt-%, more preferably, no more than 5 wt-%, and most preferably, no more than 2.5 wt-%, based on the weight of the composition.

### Redox Initiator Systems

In certain embodiments, the compositions of the present invention are chemically hardenable, i.e., the compositions contain a chemically hardenable component and a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements, resin-modified glass ionomer cements, redox cure systems, and combinations thereof.

The chemically hardenable compositions may include redox cure systems that include a hardenable component (e.g., an ethylenically unsaturated polymerizable component) and redox agents that include an oxidizing agent and a reducing agent. Suitable hardenable components, redox agents, optional acid-functional components, and optional fillers that are useful in the present invention are described in U.S. Pat. No. 6,982,288 (Mitra et al.) and U.S. Pat. No. 7,173,074 (Mitra et al.).

The reducing and oxidizing agents should react with or otherwise cooperate with one another to produce free-radicals capable of initiating polymerization of the resin system (e.g., the ethylenically unsaturated component). This type of cure is a dark reaction, that is, it is not dependent on the presence of light and can proceed in the absence of light. The reducing and oxidizing agents are preferably sufficiently shelf-stable and free of undesirable colorization to permit their storage and use under typical dental conditions. They should be sufficiently miscible with the resin system (and preferably water-soluble) to permit ready dissolution in (and discourage separation from) the other components of the hardenable composition.

Useful reducing agents include ascorbic acid, ascorbic acid derivatives, and metal complexed ascorbic acid compounds as described in U.S. Pat. No. 5,501,727 (Wang et al.); amines, especially tertiary amines, such as 4-tert-butyl dimethylaniline; aromatic sulfinic salts, such as p-toluenesulfinic salts and benzenesulfinic salts; thioureas, such as 1-ethyl-2-thiourea, tetraethyl thiourea, tetramethyl thiourea, 1,1-dibutyl thiourea, and 1,3-dibutyl thiourea; and mixtures thereof. Other secondary reducing agents may include cobalt (II) chloride, ferrous chloride, ferrous sulfate, hydrazine, hydroxylamine (depending on the choice of oxidizing agent), salts of a dithionite or sulfite anion, and mixtures thereof. Preferably, the reducing agent is an amine.

Suitable oxidizing agents will also be familiar to those skilled in the art, and include but are not limited to persulfuric acid and salts thereof, such as sodium, potassium, ammonium, cesium, and alkyl ammonium salts. Additional oxidizing agents include peroxides such as benzoyl peroxides, hydroperoxides such as cumyl hydroperoxide, t-butyl hydroperoxide, and amyl hydroperoxide, as well as salts of transition metals such as cobalt (III) chloride and ferric chloride, cerium (IV) sulfate, perboric acid and salts thereof, permanganic acid and salts thereof, perphosphoric acid and salts thereof, and mixtures thereof.

It may be desirable to use more than one oxidizing agent or more than one reducing agent. Small quantities of transition metal compounds may also be added to accelerate the rate of redox cure. In some embodiments it may be preferred to include a secondary ionic salt to enhance the stability of the polymerizable composition as described in U.S. Pat. No. 6,982,288 (Mitra et al.).

The reducing and oxidizing agents are present in amounts sufficient to permit an adequate free-radical reaction rate. This can be evaluated by combining all of the ingredients of the hardenable composition except for the optional filler, and observing whether or not a hardened mass is obtained.

Preferably, the reducing agent is present in an amount of at least 0.01% by weight, and more preferably at least 0.1% by weight, based on the total weight (including water) of the components of the hardenable composition. Preferably, the reducing agent is present in an amount of no greater than 10% by weight, and more preferably no greater than 5% by weight, based on the total weight (including water) of the components of the hardenable composition.

Preferably, the oxidizing agent is present in an amount of at least 0.01% by weight, and more preferably at least 0.10% by weight, based on the total weight (including water) of the components of the hardenable composition. Preferably, the oxidizing agent is present in an amount of no greater than 10% by weight, and more preferably no greater than 5% by weight, based on the total weight (including water) of the components of the hardenable composition.

The reducing or oxidizing agents can be microencapsulated as described in U.S. Pat. No. 5,154,762 (Mitra et al.). This will generally enhance shelf stability of the hardenable composition, and if necessary permit packaging the reducing and oxidizing agents together. For example, through appropriate selection of an encapsulant, the oxidizing and reducing agents can be combined with an acid-functional component and optional filler and kept in a storage-stable state. Likewise, through appropriate selection of a water-insoluble encapsulant, the reducing and oxidizing agents can be combined with an FAS glass and water and maintained in a storage-stable state.

A redox cure system can be combined with other cure systems, e.g., with a hardenable composition such as described U.S. Pat. No. 5,154,762 (Mitra et al.).

### Fillers

The compositions of the present invention can optionally contain fillers. Fillers may be selected from one or more of a wide variety of materials suitable for incorporation in compositions used for dental applications, such as fillers currently used in dental restorative compositions, and the like.

The filler is preferably finely divided. The filler can have a unimodial or polymodial (e.g., bimodal) particle size distribution. Preferably, the maximum particle size (the largest dimension of a particle, typically, the diameter) of the filler is less than 20 micrometers, more preferably less than 10 micrometers, and most preferably less than 5 micrometers. Preferably, the average particle size of the filler is less than 0.1 micrometers, and more preferably less than 0.075 micrometer.

The filler can be an inorganic material. It can also be a crosslinked organic material that is insoluble in the resin system (i.e., the hardenable components), and is optionally filled with inorganic filler. The filler should in any event be nontoxic and suitable for use in the mouth. The filler can be radiopaque or radiolucent. The filler typically is substantially insoluble in water.

Examples of suitable inorganic fillers are naturally occurring or synthetic materials including, but not limited to: quartz (i.e., silica, SiO₂); nitrides (e.g., silicon nitride); glasses and fillers derived from, for example, Zr, Sr, Ce, Sb, Sn, Ba, Zn, and Al; feldspar; borosilicate glass; kaolin; talc; zirconia; titania; low Mohs hardness fillers such as those described in U.S. Pat. No. 4,695,251 (Randklev); and submicron silica particles (e.g., pyrogenic silicas such as those available under the trade designations AEROSIL, including “OX 50,” “130,” “150” and “200” silicas from Degussa Corp., Akron, Ohio and CAB-O-SIL M5 silica from Cabot Corp., Tuscola, Ill). Examples of suitable organic filler particles include filled or unfilled pulverized polycarbonates, polyepoxides, and the like.

Preferred non-acid-reactive filler particles are quartz (i.e., silica), submicron silica, zirconia, submicron zirconia, and non-vitreous microparticles of the type described in U.S. Pat. No. 4,503,169 (Randklev). Mixtures of these non-acid-reactive fillers are also contemplated, as well as combination fillers made from organic and inorganic materials.

The filler can also be an acid-reactive filler. Suitable acid-reactive fillers include metal oxides, glasses, and metal salts. Typical metal oxides include barium oxide, calcium oxide, magnesium oxide, and zinc oxide. Typical glasses include borate glasses, phosphate glasses, and fluoroaluminosilicate (“FAS”) glasses. FAS glasses are particularly preferred. The FAS glass typically contains sufficient elutable cations so that a hardened dental composition will form when the glass is mixed with the components of the hardenable composition. The glass also typically contains sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass typically is in the form of particles that are sufficiently finely divided so that they can conveniently be mixed with the other cement components and will perform well when the resulting mixture is used in the mouth.

Generally, the average particle size (typically, diameter) for the FAS glass is no greater than 12 micrometers, typically no greater than 10 micrometers, and more typically no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT, RELY X LUTING PLUS CEMENT, PHOTAC-FIL QUICK, KETAC-MOLAR, and KETAC-FIL PLUS (3M ESPE Dental Products, St. Paul, Minn.), FUJI II LC and FUJI IX (G-C Dental Industrial Corp., Tokyo, Japan) and CHEMFIL Superior (Dentsply International, York, Pa.). Mixtures of fillers can be used if desired.

The surface of the filler particles can also be treated with a coupling agent in order to enhance the bond between the filler and the resin. The use of suitable coupling agents include gamma-methacryloxypropyltrimethoxysilane, gamma-mercaptopropyltriethoxysilane, gamma-aminopropyltrimethoxysilane, and the like. Silane-treated zirconia-silica (ZrO₂—SiO₂) filler, silane-treated silica filler, silane-treated zirconia filler, and combinations thereof are especially preferred in certain embodiments.

Other suitable fillers are disclosed in U.S. Pat. No. 6,387,981 (Zhang et al.) and U.S. Pat. No. 6,572,693 (Wu et al.) as well as PCT International Publication Nos. WO 01/30305 (Zhang et al.), WO 01/30306 (Windisch et al.), WO 01/30307 (Zhang et al.), and WO 03/063804 (Wu et al.). Filler components described in these references include nanosized silica particles, nanosized metal oxide particles, and combinations thereof. Nanofillers are also described in U.S. Pat. No. 7,090,721 (Craig et al.) and U.S. Pat. No. 7,090,722 (Budd et al.); and U.S. Pat. Application Publication Nos. 2005/0252413 A1 (Kangas et al.) and 2005/0256223 A1 (Kolb et al.). These applications, in summary, describe the following nanofiller containing compositions.

For some embodiments of the present invention that include filler (e.g., dental adhesive compositions), the compositions preferably include at least 1% by weight, more preferably at least 2% by weight, and most preferably at least 5% by weight filler, based on the total weight of the composition. For such embodiments, compositions of the present invention preferably include at most 40% by weight, more preferably at most 20% by weight, and most preferably at most 15% by weight filler, based on the total weight of the composition.

For other embodiments (e.g., where the composition is a dental restorative or an orthodontic adhesive), compositions of the present invention preferably include at least 40% by weight, more preferably at least 45% by weight, and most preferably at least 50% by weight filler, based on the total weight of the composition. For such embodiments, compositions of the present invention preferably include at most 90% by weight, more preferably at most 80% by weight, even more preferably at most 70% by weight filler, and most preferably at most 50% by weight filler, based on the total weight of the composition.

### Shrinkage

Polymerizable materials typically shrink upon polymerization. A number of factors may play a role in polymerization shrinkage. For example, shrinkage may occur as the van der Waals distance between monomers are replaced by covalent bonds and the packing density of the polymers increases in comparison to that of the monomers. Composite materials that exhibit reduced polymeric shrinkage without sacrificing other beneficial properties, such as fracture toughness and aesthetics, are of interest for dental applications.

Shrinkage, or volumetric change, of dental compositions upon curing can be determined, for example, according to a procedure described in Watts, D. C., et al., Determination of Polymerization Shrinkage Kinetics in Visible-Light-Cured Materials: Methods Development. Dental Materials, October, 1991, pp. 281-287. Shrinkage determined by this method is referred to herein as “Watts Shrinkage.” Preferably the hardenable materials disclosed herein have a Watts Shrinkage of less than 1.8%.

Optional Photobleachable and/or Thermochromic Dyes

In some embodiments, compositions of the present invention preferably have an initial color remarkably different than dental structures. Color is preferably imparted to the composition through the use of a photobleachable or thermochromic dye. The composition preferably includes at least 0.001% by weight photobleachable or thermochromic dye, and more preferably at least 0.002% by weight photobleachable or thermochromic dye, based on the total weight of the composition. The composition preferably includes at most 1% by weight photobleachable or thermochromic dye, and more preferably at most 0.1% by weight photobleachable or thermochromic dye, based on the total weight of the composition. The amount of photobleachable and/or thermochromic dye may vary depending on its extinction coefficient, the ability of the human eye to discern the initial color, and the desired color change. Suitable thermochromic dyes are disclosed, for example, in U.S. Pat. No. 6,670,436 (Burgath et al.).

For embodiments including a photobleachable dye, the color formation and bleaching characteristics of the photobleachable dye varies depending on a variety of factors including, for example, acid strength, dielectric constant, polarity, amount of oxygen, and moisture content in the atmosphere. However, the bleaching properties of the dye can be readily determined by irradiating the composition and evaluating the change in color. Preferably, at least one photobleachable dye is at least partially soluble in a hardenable resin.

Exemplary classes of photobleachable dyes are disclosed, for example, in U.S. Pat. No. 6,331,080 (Cole et al.), U.S. Pat. No. 6,444,725 (Trom et al.), and U.S. Pat. No. 6,528,555 (Nikutowski et al.). Preferred dyes include, for example, Rose Bengal, Methylene Violet, Methylene Blue, Fluorescein, Eosin Yellow, Eosin Y, Ethyl Eosin, Eosin bluish, Eosin B, Erythrosin B, Erythrosin Yellowish Blend, Toluidine Blue, 4′,5′-Dibromofluorescein, and combinations thereof.

The color change in the inventive compositions is initiated by light. Preferably, the composition's color change is initiated using actinic radiation using, for example, a dental curing light which emits visible or near infrared (IR) light for a sufficient amount of time. The mechanism that initiates the color change in the compositions of the invention may be separate from or substantially simultaneous with the hardening mechanism that hardens the resin. For example, a composition may harden when polymerization is initiated chemically (e.g., redox initiation) or thermally, and the color change from an initial color to a final color may occur subsequent to the hardening process upon exposure to actinic radiation.

The change in composition color from an initial color to a final color is preferably quantified by a color test. Using a color test, a value of ΔE* is determined, which indicates the total color change in a 3-dimensional color space. The human eye can detect a color change of approximately 3 ΔE* units in normal lighting conditions. The dental compositions of the present invention are preferably capable of having a color change, ΔE*, of at least 20; more preferably, ΔE* is at least 30; most preferably ΔE* is at least 40.

### Miscellaneous Optional Additives

Optionally, compositions of the present invention may contain solvents (e.g., alcohols (e.g., propanol, ethanol), ketones (e.g., acetone, methyl ethyl ketone), esters (e.g., ethyl acetate), other nonaqueous solvents (e.g., dimethylformamide, dimethylacetamide, dimethylsulfoxide, 1-methyl-2-pyrrolidinone)), and water.

If desired, the compositions of the invention can contain additives such as indicators, dyes, pigments, inhibitors, accelerators, viscosity modifiers, wetting agents, buffering agents, stabilizers, and other similar ingredients that will be apparent to those skilled in the art. Viscosity modifiers include the thermally responsive viscosity modifiers (such as PLURONIC F-127 and F-108 available from BASF Wyandotte Corporation, Parsippany, N.J.) and may optionally include a polymerizable moiety on the modifier or a polymerizable component different than the modifier. Such thermally responsive viscosity modifiers are described in U.S. Pat. No. 6,669,927 (Trom et al.) and U.S. Pat. Application Publication No. 2004/0151691 (Oxman et al.).

Additionally, medicaments or other therapeutic substances can be optionally added to the dental compositions. Examples include, but are not limited to, fluoride sources, whitening agents, anticaries agents (e.g., xylitol), calcium sources, phosphorus sources, remineralizing agents (e.g., calcium phosphate compounds), enzymes, breath fresheners, anesthetics, clotting agents, acid neutralizers, chemotherapeutic agents, immune response modifiers, thixotropes, polyols, anti-inflammatory agents, antimicrobial agents (in addition to the antimicrobial lipid component), antifungal agents, agents for treating xerostomia, desensitizers, and the like, of the type often used in dental compositions. Combination of any of the above additives may also be employed. The selection and amount of any one such additive can be selected by one of skill in the art to accomplish the desired result without undue experimentation.

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

### Examples

Unless otherwise noted, all solvents and reagents were or can be obtained from Sigma-Aldrich Corp., St. Louis, Mo.

As used herein,

“IEM” refers to 2-isocyanatoethyl methacrylate;

“BHT” refers to 2,6-di-tert-butyl-4-methylphenol;

“BisGMA” refers to 2,2-bis[4-(2-hydroxy-3-methacryloyloxypropoxy)phenyl]propane;

“UDMA” refers to diurethane dimethacrylate, available from Monomer-Polymer & Dajac Labs, Inc., Feasterville, Pa.);

“CPQ” refers to camphorquinone;

“EDMAB” refers to ethyl 4-(N,N-dimethylamino)benzoate;

“DPIHFP” refers to diphenyl iodonium hexafluorophosphate;

“FILLER A” refers to a silane-treated nano-sized silica having a nominal particle size of approximately 20 nanometers, prepared according to the procedure for FILLER F in U.S. Pat. Application Publication No. 2005/0252413 (Kangas et al.);

“FILLER B” refers to silane-treated zirconia-silica filler prepared as described in U.S. Pat. No. 6,730,156 (Windisch et al.);

“TrisMAS” refers to the product of Example 1;

“TrisMAC” refers to the product of Example 4;

“TrisMAP” refers to the product of Example 2;

“CAMP” refers to the product of Preparative Example 2;

“CAMUDMA” refers to the product of Preparative Example 3.

### Determination of Diametral Tensile Strength (DTS) and Compressive Strength (CS)

Diametral tensile strength and compressive strength were determined according to ANSI/ADA specification No. 27 (1993). Each composition was heated to 85° C. for 30 minutes, injected into a glass tube having an inside diameter of 4 millimeters, and then the packed tube was then capped with silicone rubber plugs. The composition was then compressed axially at approximately 0.28 MPa (approximately 40.62 pounds per square inch) for 5 minutes. While under compression, the sample was then light cured for 90 seconds by exposure to a Model XL 1500 dental curing light (manufactured by 3M ESPE Dental Products, St. Paul, Minn.) and was then irradiated for 90 seconds in a Model UNIXS light curing box (manufactured by Heraeus Kulzer, Hanau, Germany). The cured sample was allowed to stand at 37° C. and at least 90% relative humidity for approximately one hour. The cured sample was then cut crosswise using a diamond saw to afford discs having a length of approximately 2 millimeters (for the DTS test) or approximately 8 millimeters (for the CS test). The discs were stored in distilled water at 37° C. for 24 hours prior to testing. The DTS and CS tests were carried out on a Model 4505 Instron tester (manufactured by Instron Corp., Norwood, Mass.) with a 10-kilonewton (kN) load cell and at a crosshead speed of 1 meter per minute. Five discs of each cured composition were prepared and tested for DTS. Six discs of each cured composition were prepared and tested for CS.

### Determination of Shrinkage (Watts Shrinkage)

The shrinkage (volumetric change) of the dental compositions on curing was determined on samples weighing 0.090 g according to the procedure described in Watts, D. C., et al., Determination of Polymerization Shrinkage Kinetics in Visible-Light-Cured Materials: Methods Development. Dental Materials, October, 1991, pp. 281-287. The average shrinkage of three samples of each dental composition is reported in the Examples.

### Barcol Hardness

Samples of each dental composition were cured by placing each sample in a poly(tetrafluoroethylene) mold having a thickness of 2.5 millimeters, covering each open side of the mold with polyester film and then covering each polyester film with a glass slide. Then the dental compositions were each cured using a dental curing light (available under the trade designation EPILAR FreeLight 2 from 3M ESPE Dental Products, St. Paul, Minn.) for 30 seconds. The glass slides and then the polyester films were removed and the Barcol hardness of the top (i.e., the side that was irradiated with the EPILAR FreeLight 2) and bottom (i.e., the side that was not irradiated with the EPILAR FreeLight 2) of each cured dental composition was evaluated using a Model GYZJ 934-1 hand-held portable hardness tester, available from InstruCon, Inc., Rockford, Ill., according to the directions provided by the manufacturer. In the Examples, the Barcol hardness of the bottom and top of the sample are given as the “B” and “T” hardness, respectively.

### Depth of Cure (DC)

The depth of cure (DC) of each of the cured dental compositions was determined by filling a 10 millimeter mold cavity with the dental composition, covering the top and bottom of the mold with sheets of polyester film, pressing the sheets to provide a level dental composition surface, placing the filled mold on a white background surface, irradiating the dental composition for 20 seconds using a dental curing light (Visilux 2, 3M ESPE Dental Products, St. Paul, Minn.), separating the polyester films from each side of the mold, gently removing(by scraping) material from the bottom of the sample (i.e., the side that was not irradiated with the dental curing light), and measuring the thickness of the remaining material in the mold.

### Preparative Example 1

### Preparation of 7-Methylene-1,5-dithiacyclooctan-3-ol

7-Methylene-1,5-dithiacyclooctan-3-ol was prepared by the reaction of 3-choro-2-(chloromethyl)-1-propene (available from Secant Chemicals Inc., Winchendon, Mass.) and 1,3-dibromo-2-propanol according to the procedure described in Evans, R., et al., Free-Radical Ring-Opening Polymerization of Cyclic Allylic Sulfides. 2. Effect of Substituents on Seven- and Eight-Membered Ring Low Shrink Monomers., Macromolecules, 2000, vol. 33, pp. 6722-6731.

### Preparative Example 2

### Preparation of CAMP

mono-2-Methacryloyloxyethyl phthalate (9.4 g) and the product of Preparative Example 1 (5.38 g) were combined in methylene chloride (50 mL) in a 3-neck flask equipped with a magnetic stirring bar and connected to a source of nitrogen gas. 4-Dimethylaminopyridine (0.4 g) was added to the flask and the resultant mixture was magnetically stirred and cooled in an ice bath for approximately 20 minutes. To the cooled solution there was added dropwise over approximately one hour a solution of dicylcohexylcarbodiimide (6.95 g) in methylene chloride (50 mL). The flask was left in the ice bath and the resultant mixture was then stirred for one hour and then at room temperature overnight. The precipitated solid was removed by vacuum filtration using a Buchner funnel with diatomaceous earth as a filtration aid. The filtrate was then washed once with 0.1 N HCl (100 mL), once with 5 weight percent aqueous NaOH (100 mL), and once with water (100 mL). The organic layer was dried and concentrated under vacuum to afford the product as a slightly cloudy viscous liquid (11.4 g).

### Preparative Example 3

### Preparation of CAMUDMA

2,4,4-Trimethyl-1,6-diisocyanatohexane (17.6 g) was dissolved in methylene chloride (100 mL) in a 3-neck flask equipped with a magnetic stirring bar and connected to a source of nitrogen gas. To the flask there was then sequentially added dibutyltin dilaurate (10 drops) and the product of Preparative Example 1 (14.8 g), the latter added in small aliquots. The resultant mixture was stirred at room temperature for approximately 6 hours. 2-Hydroxyethyl methacrylate (10.92 g) was added dropwise to the mixture and then the mixture was magnetically stirred overnight at room temperature. Methanol (10 mL) was added to the mixture and after 0.5 hour the volatile components were removed under reduced pressure to afford the product as a colorless viscous liquid.

### Example 1

### Preparation of TrisMAS

A 3-neck flask, fitted with a mechanical stirrer, an addition funnel, and a source of nitrogen gas, was charged with bisGMA (273.8 g), mono-2-(methacryloyloxyethyl)succinate (122.94 g), 4-(dimethylamino)pyridine (6.4 g), and ethyl acetate (400 mL). The mixture was mechanically stirred while the flask was cooled in a bath of ice. After approximately 20 minutes, a solution of dicyclohexylcarbodiimide (111 g) in ethyl acetate (150 mL) was added dropwise to the cold stirring mixture. The cold mixture was then stirred for approximately one hour, after which time it was allowed to warm to room temperature. The mixture was then stirred overnight at room temperature. The mixture was then vacuum filtered and the filtrate was washed with IN aqueous HCl (200 mL). The organic phase was dried over anhydrous sodium sulfate and was concentrated under vacuum. The resultant viscous liquid was vacuum filtered using a fritted glass funnel to afford the product as a clear colorless liquid.

### Example 2

### Preparation of TrisMAP

A 3-neck flask, fitted with a mechanical stirrer, an addition funnel, and a source of nitrogen gas, was charged with bisGMA (342.5 g), mono-2-(methacryloyloxyethyl)phthalate (181 g), 4-(dimethylamino)pyridine (6.4 g), and ethyl acetate (400 mL). The mixture was mechanically stirred while the flask was cooled in a bath of ice. After approximately 20 minutes, a solution of dicyclohexylcarbodiimide (136 g) in ethyl acetate (150 mL) was added dropwise to the cold stirring mixture. The cold mixture was then stirred for approximately one hour, after which time it was allowed to warm to room temperature. The mixture was then stirred overnight at room temperature. The mixture was then vacuum filtered and the filtrate was washed with 1N aqueous HCl (200 mL). The organic phase was dried over anhydrous sodium sulfate and was concentrated under vacuum. The resultant viscous liquid was vacuum filtered using a fritted glass funnel to afford the product as a clear colorless liquid.

### Example 3

A 500 mL 3-neck flask, fitted with a mechanical stirrer, a reflux condenser, and a source of dry air, was charged with bisGMA (169 g). The flask was heated to approximately 50° C., and then caprolactone (74 g), tin (II) octanoate (10 drops), and BHT (0.15 g) were added to the flask. The flask was heated to 125° C. and the mixture was mechanically stirred overnight. The flask was then allowed to cool to afford the 1:1 adduct.

### Example 4

### Preparation of TrisMAC

A sample of the 1:1 adduct of the product of Example 3 (42.4 g) was transferred to a glass jar. Dibutyltin dilaurate (10 drops) was added to the adduct, and the mixture was stirred by hand with a poly(tetrafluoroethylene) rod. To the viscous liquid there was added IEM (8.8 g) in small portions. The mixture was stirred by hand after the addition of each portion of IEM. The jar containing the mixture was allowed to stand at room temperature overnight to afford the product. Analysis of the product using Fourier transform infrared spectrometry indicated no absorbances attributable to the isocyanate group.

### Example 5

A 500 mL 3-neck flask, fitted with a mechanical stirrer, a reflux condenser, and a source of dry air, was charged with bisGMA (296 g). The flask was heated to approximately 50° C., and then caprolactone (198 g), tin (II) octanoate (10 drops), and BHT (0.15 g) were added to the flask. The flask was heated to 150° C. and the mixture was mechanically stirred overnight. The flask was then allowed to cool to afford the 1:1.5 adduct.

### Example 6

A sample of the 1:1.5 adduct of the product of Example 5 (42 g) was transferred to a glass jar. Dibutyltin dilaurate (10 drops) was added to the adduct, and the mixture was stirred by hand with a poly(tetrafluoroethylene) rod. To the viscous liquid there was added IEM (7.5 g) in small portions. The mixture was stirred by hand after the addition of each portion of IEM. The jar containing the mixture was allowed to stand at room temperature overnight to afford the product. Analysis of the product using Fourier transform infrared spectrometry indicated no absorbances attributable to the isocyanate group.

### Examples 7-13

### Dental Compositions and Properties

The hardenable dental resin compositions of Examples 7-13 were prepared using Samples A-G, each having the composition indicated in Table 1. In Table 1, all percentages are weight percentages, and “- - - ” means that the component was not included in the polymerizable resin sample. Each mixture of Samples A-G was heated to approximately 65° C. while the components were mixed. Samples A-G were used to prepare the hardenable dental resin compositions of Examples 7-13, respectively. A weighed portion of each mixture was combined with a weighed portion of a mixture of 9 weight percent Filler A and 91 weight percent Filler B in a model MAX 20 plastic mixing cup (available from FlackTek, Inc., Landrum, S.C.) to give the hardenable dental resin compositions of Examples 7-13 having the following weight percentages of filler mixture: 76.25% (Example 7), 76.59% (Example 8), 75.57% (Example 9), 76.17% (Example 10), 76.0% (Example 11), 77.0% (Example 12), and 78.0% (Example 13). Each dental composition was then mixed for one minute using a Model DAC 150 FVZ SpeedMixer (manufactured by FlackTek, Inc., Landrum, S.C.) at 3000 rpm. Each dental composition was then heated to 85° C. for approximately 30 minutes and was then mixed two times more, using the SpeedMixer for one minute at 3000 rpm.

TABLE 1
Compositions of Samples A-G of Examples 7-13.

| | Sample | | | | | | |
|---|---|---|---|---|---|---|---|
| Component | A | B | C | D | E | F | G |
| Example 3 | 98.23% | — | — | — | 78.85% | 78.85% | 78.85% |
| Example 4 | — | — | 98.23% | — | — | — | — |
| Example 5 | — | 98.23% | — | — | — | — | — |
| Example 6 | — | — | — | 98.23% | — | — | — |
| UDMA | — | — | — | — | 19.67% | 19.67% | 19.67% |
| CPQ | 0.17% | 0.17% | 0.17% | 0.17% | 0.17% | 0.17% | 0.17% |
| EDMAB | 1.0% | 1.0% | 1.0% | 1.0% | 0.98% | 0.98% | 0.98% |
| DPIHFP | 0.5% | 0.5% | 0.5% | 0.5% | 0.5% | 0.5% | 0.5% |
| BHT | 0.1% | 0.1% | 0.1% | 0.1% | 0.11% | 0.11% | 0.11% |

The shrinkage, diametral tensile strength (DTS), bottom (B) and top (T) Barcol hardness, and depth of cure (DC) of each dental composition of Examples 7-13 and of the dental composition of Comparative Example 1 (a commercially-available dental composition available under the trade designation FILTEK SUPREME UNIVERSAL RESTORATIVE from 3M ESPE Dental Products, St. Paul, Minn.) were evaluated as described above. The data are given in Table 2. In Table 2, “NM” means that the data was not obtained.

TABLE 2
Properties of Dental Compositions of Examples 7-13 and
Comparative Example 1.

| Example | Shrinkage | DTS | Barcol (B/T) | DC |
|---|---|---|---|---|
| 7 | 1.52% | 63.4 MPa | 67/67 | 3.38 mm |
| 8 | 1.46% | 57.8 MPa | 62/62 | 2.96 mm |
| 9 | 1.55% | 78.6 MPa | 77/79 | 3.27 mm |
| 10 | 1.51% | 74.3 MPa | 75/75 | 2.96 mm |
| 11 | 1.68% | 67.0 MPa | NM | 3.35 mm |
| 12 | 1.60% | 68.5 MPa | NM | 3.08 mm |
| 13 | 1.57% | 60.9 MPa | NM | 3.13 mm |
| Comparative 1 | 1.84% | 75.5 MPa | 84/83 | 3.27 mm |

### Examples 14-17

### Preparation of Dental Compositions including TrisMAP

The hardenable dental resin compositions of Examples 14-17 were prepared in a manner similar to those of Examples 7-13. Samples H-K, having the compositions given in Table 3, were prepared. In Table 3, all percentages are weight percentages. Samples H-K were used to prepare the hardenable dental resin compositions of Examples 14-17, respectively. A weighed portion of each mixture was combined with a weighed portion of the filler mixture, as described above, to give the dental compositions of Examples 14-17 having the following weight percentages of filler mixture: 72.0% (Example 14), 74.0% (Example 15), 76.0% (Example 16), and 78.0% (Example 17).

TABLE 3
Compositions of Samples H-K of Examples 14-17.

Sample

| Component | H | I | J | K |
|---|---|---|---|---|
| TrisMAP | 22.0% | 20.45% | 18.87% | 17.30% |
| UDMA | 5.5% | 5.11% | 4.72% | 4.33% |
| CPQ | 0.06% | 0.05% | 0.05% | 0.04% |
| EDMAB | 0.28% | 0.26% | 0.24% | 0.22% |
| DPIHFP | 0.14% | 0.13% | 0.12% | 0.11% |

The shrinkage, diametral tensile strength (DTS), and depth of cure (DC) of each dental composition of Examples 14-17 and of the dental composition of Comparative Example 2 (a commercially-available dental composition available under the trade designation FILTEK SUPREME UNIVERSAL RESTORATIVE from 3M ESPE Dental Products, St. Paul, Minn.) were evaluated as described above. The data are given in Table 4. In Table 4, “NM” means that the data was not obtained.

TABLE 4
Properties of Dental Compositions of Examples 14-17.

| Example | Shrinkage | DTS | DC VISILUX2 | DC FreeLight2 |
|---|---|---|---|---|
| 14 | 1.51% | 73 MPa | 3.46 | 4.84 |
| 15 | 1.50% | 76 MPa | 3.68 | 4.5 |
| 16 | 1.42% | 75 MPa | 3.8 | 4.6 |
| 17 | NM | 74 MPa | 3.52 | 4.75 |
| Comparative | 1.82% | 75.6 MPa | 3.58 | 4.4 |
| 2 | | | | |

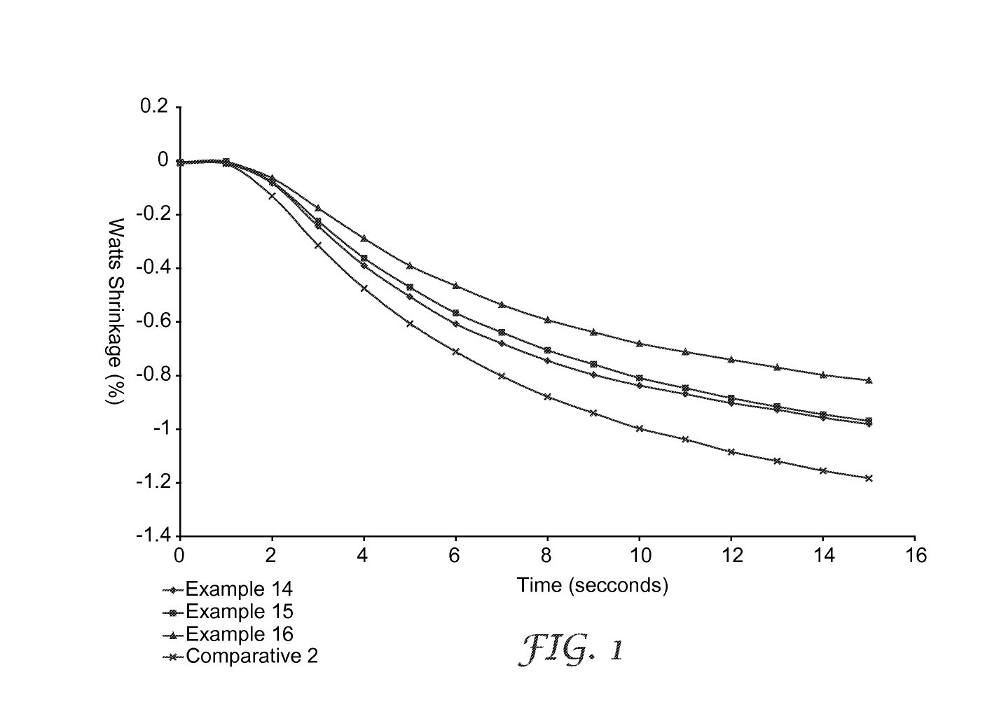

Evaluation of shrinkage of the compositions of Examples 14-16 and Comparative Example 2 included data collection each second from 0 to 15 seconds during the cure. These data are shown in Table 5 and in FIG. 1.

TABLE 5
Watts Shrinkage Data for Compositions of Examples 14-16 and
Comparative Example 2.

| Time (sec) | Example 14 | Example 15 | Example 16 | Comparative 2 |
|---|---|---|---|---|
| 0 | −0.009% | −0.004% | −0.005% | −0.007% |
| 1 | −0.010% | −0.002% | −0.007% | −0.010% |
| 2 | −0.083% | −0.078% | −0.064% | −0.131% |
| 3 | −0.243% | −0.224% | −0.176% | −0.315% |
| 4 | −0.390% | −0.362% | −0.289% | −0.475% |
| 5 | −0.506% | −0.471% | −0.391% | −0.606% |
| 6 | −0.608% | −0.567% | −0.465% | −0.711% |
| 7 | −0.680% | −0.639% | −0.536% | −0.802% |
| 8 | −0.745% | −0.706% | −0.593% | −0.879% |
| 9 | −0.797% | −0.758% | −0.638% | −0.94% |
| 10 | −0.837% | −0.809% | −0.681% | −0.998% |
| 11 | −0.869% | −0.847% | −0.712% | −1.038% |
| 12 | −0.903% | −0.884% | −0.741% | −1.085% |
| 13 | −0.928% | −0.916% | −0.770% | −1.119% |
| 14 | −0.957% | −0.945% | −0.798% | −1.155% |
| 15 | −0.981% | −0.969% | −0.818% | −1.183% |

### Examples 18-24

### Preparation of Dental Compositions

The hardenable dental resin compositions of Examples 18-24 were prepared in a manner similar to those of Examples 7-13. Polymerizable resin Samples L-R, having the compositions given in Table 6, were prepared. In Table 6, all percentages are weight percentages, and “- - - ” means that the component was not included in the polymerizable resin sample. Samples L-R were used to prepare the dental compositions of Examples 18-24, respectively. A weighed portion of each mixture was combined with a weighed portion of the filler mixture, as described above, to give the dental compositions of Examples 18-24 each having 75.0 weight percent of filler mixture.

TABLE 6
Compositions of Samples L-R of Examples 18-24.

| | Sample | | | | | | |
|---|---|---|---|---|---|---|---|
| Component | L | M | N | O | P | Q | R |
| Example 4 | 24.56% | 19.64% | 17.24% | 14.76% | — | — | — |
| Example 1 | — | — | — | — | 17.20% | 24.54% | 20.86% |
| CAMP | — | 3.27% | 4.95% | 6.59% | 7.37% | — | 3.68% |
| CAMUDMA | — | 1.62% | 2.45% | 3.26% | — | — | — |
| CPQ | 0.04% | 0.04% | 0.04% | 0.04% | 0.05% | 0.05% | 0.05% |
| EDMAB | 0.25% | 0.25% | 0.25% | 0.25% | 0.25% | 0.25% | 0.25% |
| DPIHFP | 0.12% | 0.12% | 0.12% | 0.12% | 0.13% | 0.13% | 0.13% |
| BHT | 0.02% | 0.02% | 0.02% | 0.02% | — | — | — |

The shrinkage, diametral tensile strength (DTS), and depth of cure (DC) of each dental composition of Examples 18-24 were evaluated as described above. The data are given in Table 7.

TABLE 7
Properties of Dental Compositions of Examples 18-24.

| Example | Shrinkage | DTS | DC |
|---|---|---|---|
| 18 | 1.44% | 78.0 MPa | 3.51 mm |
| 19 | 1.53% | 77.0 MPa | 2.65 mm |
| 20 | 1.57% | 80.0 MPa | 2.30 mm |
| 21 | 1.56% | 77.0 MPa | 2.15 mm |
| 22 | 1.67% | 78.8 MPa | N.D. |
| 23 | 1.56% | 73.1 MPa | N.D. |
| 24 | 1.58% | 75.6 MPa | N.D. |

N.D. means not determined.

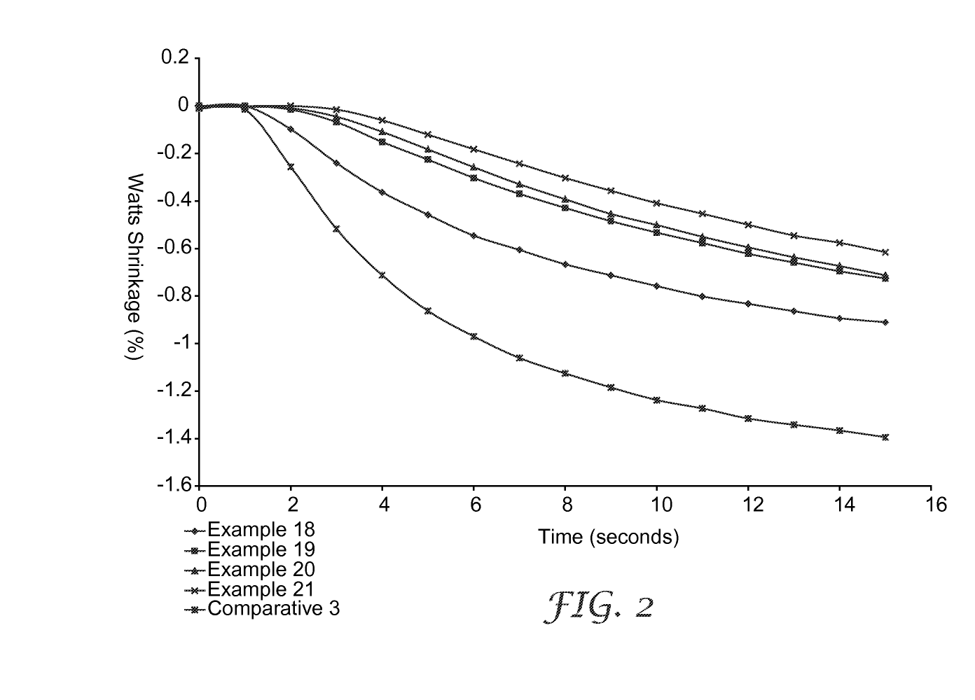

Evaluation of shrinkage of the compositions of Examples 18-21 and Comparative Example 3 (a commercially-available dental composition available under the trade designation FILTEK SUPREME UNIVERSAL RESTORATIVE from 3M ESPE Dental Products, St. Paul, Minn.) included data collection each second from 0 to 15 seconds during the cure. These data are shown in Table 8 and in FIG. 2.

TABLE 8
Watts Shrinkage Data for Compositions of Examples 18-21 and
Comparative Example 3.

| Time | Example | Example | Example | | |
|---|---|---|---|---|---|
| (sec) | 18 | 19 | 20 | Example 21 | Comparative 3 |
| 0 | 0% | 0% | −0.005% | 0% | −0.011% |
| 1 | 0% | 0% | −0.004% | −0.001% | −0.014% |
| 2 | −0.098% | −0.015% | −0.009% | 0% | −0.256% |
| 3 | −0.240% | −0.068% | −0.045% | −0.015% | −0.518% |
| 4 | −0.363% | −0.152% | −0.109% | −0.060% | −0.713% |
| 5 | −0.458% | −0.226% | −0.183% | −0.121% | −0.863% |
| 6 | −0.546% | −0.303% | −0.258% | −0.182% | −0.970% |
| 7 | −0.606% | −0.370% | −0.330% | −0.243% | −1.061% |
| 8 | −0.667% | −0.429% | −0.393% | −0.303% | −1.126% |
| 9 | −0.713% | −0.485% | −0.455% | −0.357% | −1.185% |
| 10 | −0.758% | −0.533% | −0.501% | −0.409% | −1.238% |
| 11 | −0.802% | −0.577% | −0.551% | −0.454% | −1.273% |
| 12 | −0.833% | −0.622% | −0.595% | −0.500% | −1.316% |
| 13 | −0.864% | −0.659% | −0.637% | −0.546% | −1.342% |
| 14 | −0.894% | −0.696% | −0.674% | −0.576% | −1.366% |
| 15 | −0.910% | −0.726% | −0.713% | −0.616% | −1.394% |

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A method of preparing a hardenable material, the method comprising:
combining components comprising 2,2-bis[4-(2-hydroxy-3-methacryloxypropoxy)phenyl]propane (BisGMA) and at least one lactone under conditions sufficient to chain extend at least one of the two BisGMA hydroxyl groups with a ring-opened lactone; wherein the chain-extended BisGMA is non-crystalline.

2-6. (canceled)

7. The method of claim 1 wherein the at least one lactone comprises ε-caprolactone; and wherein the chain-extended BisGMA comprises one chain-extended BisGMA hydroxyl group.

8. (canceled)

9. The method of claim 7 wherein the one chain-extended BisGMA hydroxyl group comprises at most eight ring-opened ε-caprolactone molecules.

10-11. (canceled)

12. The method of claim 1 wherein the at least one lactone comprises ε-caprolactone; and wherein the chain-extended BisGMA comprises two chain-extended BisGMA hydroxyl groups.

13-14. (canceled)

15. The method of claim 12 wherein each of the two BisGMA chain-extended hydroxyl groups comprises at most four ring-opened ε-caprolactone molecules.

16. (canceled)

17. The method of claim 1 further comprising (meth)acryloylating at least one hydroxyl group.

18-28. (canceled)

29. A hardenable material comprising a non-crystalline, chain-extended 2,2-bis[4-(2-hydroxy-3-methacryloxypropoxy)phenyl]propane (BisGMA), wherein the chain extended BisGMA comprises at least one of the two BisGMA hydroxyl groups chain-extended with at least one ring-opened lactone.

30-31. (canceled)

32. A hardenable composition comprising a hardenable material according to claim 29.

33-41. (canceled)

42. A method of treating an oral surface, the method comprising:
applying a hardenable composition according to claim 32 to the oral surface; and
hardening the hardenable composition.

43-44. (canceled)

45. A method of preparing a hardenable material, the method comprising:
combining components comprising 2,2-bis[4-(2-hydroxy-3-methacryloxypropoxy)phenyl]propane (BisGMA) and at least one methacrylate-functional carboxylic acylating agent under conditions sufficient to acylate at least one of the two BisGMA hydroxyl groups.

46. The method of claim 45 wherein the at least one carboxylic acylating agent is selected from the group consisting of methacrylate-functional carboxylic acids, methacrylate-functional carboxylic acid halides, methacrylate-functional carboxylic acid anhydrides, methacrylate-functional active esters of carboxylic acids, and combinations thereof.

47. The method of claim 46 wherein the methacrylate-functional carboxylic acid comprises a mono-ester of a dicarboxylic acid.

48. The method of claim 47 wherein the methacrylate-functional carboxylic acid is selected from the group consisting of mono-2-(methacryloyloxyethyl)-succinate, mono-2-(methacryloyloxyethyl)phthalate, mono-3-(methacryloylxyethyl)glutarate, mono-4-(methacryloyloxyethyl)adipate, and combinations thereof.

49-56. (canceled)

57. A hardenable material comprising an acylated 2,2-bis[4-(2-hydroxy-3-methacryloxypropoxy)phenyl]propane (BisGMA), wherein the acylated BisGMA comprises at least one BisGMA hydroxyl group acylated with at least one methacrylate-functional carboxylic acylating agent.

58. (canceled)

59. The hardenable material of claim 57 wherein the at least one carboxylic acylating agent comprises a methacrylate-functional carboxylate selected from the group consisting of mono-2-(methacryloyloxyethyl)-succinate, mono-2-(methacryloyloxyethyl)phthalate, mono-3-(methacryloylxyethyl)glutarate, mono-4-(methacryloyloxyethyl)adipate, and combinations thereof.

60. A hardenable composition comprising a hardenable material according to claim 57.

61-69. (canceled)

70. A method of treating an oral surface, the method comprising:
applying a hardenable composition according to claim 60 to the oral surface; and
hardening the hardenable composition.

71-72. (canceled)

73. A compound of the formula 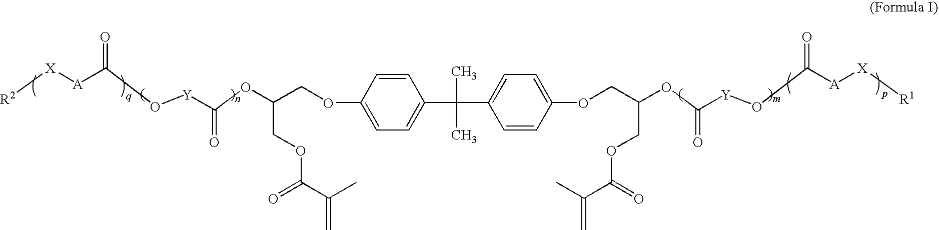
wherein:
each X independently represents an oxygen atom (O) or a nitrogen atom (N); Y and A each independently represent an organic group, with the proviso that Y does not represent —NHCH₂CH₂— if (i) p=0 and R¹ represents —C(O)C(CH₃)═CH₂, and/or (ii) q=0 and R² represents —C(O)C(CH₃)═CH₂;
m=1 to 5;
n=0 to 5;
p and q are independently 0 or 1; and
R¹ and R² each independently represent H, —C(O)CH═CH₂, or —C(O)C(CH₃)═CH₂.

74-83. (canceled)

84. A hardenable composition comprising a compound according to claim 73.

85-89. (canceled)

90. A method of treating an oral surface, the method comprising:
applying a hardenable composition according to claim 84 to the oral surface; and
hardening the hardenable composition.

91-92. (canceled)
